(19) United States
(12) Patent Application Publication
(10) Pub. No.: US 2023/0330139 A1
(43) Pub. Date: Oct. 19, 2023

(54) CAR T-CELLS COMPRISING AN ANTI CD33, AN ANTI CLL1 AND AT LEAST ONE FURTHER CAR ANTI CD123 AND/OR FTL3

(71) Applicant: AUTOLUS LIMITED, London (GB)

(72) Inventors: Martin Pulé, London (GB); Shaun Cordoba, London (GB); Simon Thomas, London (GB); Shimobi Onuoha, London (GB); Alexander Kinna, London (GB); Mathieu Ferrari, London (GB)

(21) Appl. No.: 17/268,328

(22) PCT Filed: Aug. 13, 2019
(86) PCT No.: PCT/GB2019/052275

§ 371 Date: Feb. 12, 2021

(30) Foreign Application Priority Data
Aug. 13, 2018 (GB) 1813178.9

(51) Int. Cl. A61K 35/17; C07K 16/28; A61P 35/02

(52) U.S. Cl. CPC A61K 35/17; C07K 16/2803; C07K 16/2851; C07K 16/2866; A61P 35/02

(57) ABSTRACT

The present disclosure provides a cell comprising: an anti-CD33 chimeric antigen receptor (CAR); an anti-CLL1 CAR; and an anti- CD123 and/or anti- CAR FLT3 CAR. The cell can be used in the treatment of a disease such as acute myeloid leukemia (AML).

### FIELD OF THE INVENTION

The present invention relates to a cell which expresses multiple chimeric antigen receptors (CARs). The cell targets multiple antigens characteristic of acute myeloid leukemia (AML).

### BACKGROUND TO THE INVENTION

### Acute Myeloid Leukemia

Acute myeloid leukemia (AML) is a heterogeneous disease characterized by the uncontrolled clonal proliferation of myeloid precursors in the bone marrow and blood, resulting in accumulation of leukemic blasts and severe impairment of normal hematopoiesis. AML is the most common acute leukemia in adults and has the highest death rate of all leukemias. An estimated 20830 people in the Unites States were predicted be diagnosed with AML and 10460 deaths were projected to occur from AML in 2015. Gains in long-term survival in AML over the last decade have remained modest.

Current induction chemotherapy can produce initial complete remission in almost 70% of young adult patients. However, 43% of patients will eventually relapse, and 18% never attain complete remission at frontline induction treatment. The 5-year overall survival rate for patients with AML after the first relapse ranges from 7 to 12%. Allogeneic hematopoietic stem cell transplantation (alloHSCT) provides the best chance to cure a patient with relapsed or refractory AML. It is the preferred treatment route following a second remission and can lead to 5-year disease-free survival in 40-50% of patients.

Using current treatment strategies, the second complete remission rate is achieved in only about half of relapsed patients who previously attained a complete remission that lasted longer than 6 months and is only 20% or fewer of patients with primary refractory disease and those with an initial complete remission lasting less than 6 months. In addition, the considerable complications of conventional salvage chemotherapy may worsen the performance status and organ function of the patient and decrease the chance of a successful allo HSCT.

There is therefore a need for improved therapeutic approaches to treat AML.

### Chimeric Antigen Receptors

Chimeric antigen receptors (CARs) graft the specificity of a monoclonal antibody onto the effector function of a T-cell. CAR T-cell therapy directed against CD19 has been highly effective in B-cell malignancies, although CD19 negative escape is a cause of relapse in a considerable portion of patients.

CAR T-cell therapy against AML is in early clinical testing. Despite several preclinical studies demonstrating cytotoxic potential of various AML antigen-targeting CAR T cells, translation to the clinic has been slow. This highlights inherent challenges in developing CAR-related treatment strategies for patients with AML. To date, a small number of patients with relapsed/refractory AML have been treated with CAR T-cell immunotherapies via early phase clinical trials, as shown Table 1.

TABLE 1
Current phase I clinical trials of CAR T-cell immunotherapy
for patients with relapsed/refractory AML

| AML target | ClinicalTrials.gov | |
|---|---|---|
| antigen | identifier | Site of conduction |
| CD33 | NCT03126864 | MDACC, USA |
| | NCT01864902 | PLA Hospital, |
| | | Beijing, China |
| | NCT02799680 | AHCAMMS, |
| | | Beijing, China |
| CD38 | NCT03222674 | Geno-Immune |
| | | Medical Institute, |
| | | Shenzhen, China |
| CD56 | NCT03222674 | Geno-Immune |
| | | Medical Institute, |
| | | Shenzhen, China |
| CD117 | NCT03222674 | Geno-Immune |
| | | Medical Institute, |
| | | Shenzhen, China |
| CD123 | NCT02159495 | City of Hope, USA |

### DESCRIPTION OF THE FIGURES

FIG. 1—Schematic diagram illustrating hematopoiesis in humans

FIG. 2—Different binding domain formats of chimeric antigen receptors (a) Fab CAR format; (b) dAb CAR format; (c) scFv CAR format

FIG. 3—Expression of aCD33 CARs on primary T cells derived from three healthy donors. CARs transduced with T cells were stained with RQR8 Marker gene. FACS plots were pre-gated for live cell population by using eFluor780.

FIG. 4—Transduction expression of CAR constructs used in aCD123-VHH CAR screening and validation assays. PBMCs were stained separately with an anti-CD34-PE antibody (QBend10) or soluble human CD123 ectodomain fused to mouse Fc fused to 2× StrepTag2 (Strep-PE secondary stain). All plots were pre-gated on live single cells using a viability dye (FVD-eFlour780).

FIG. 5—Transduction expression of CAR constructs used in aCLL1-VHH CAR screening and validation assays. PBMCs were stained separately with an anti-CD34-PE antibody (QBend10) or soluble human CLL1 ectodomain fused to human Fc fused to 2× StrepTag2 (Strep-PE secondary stain). All plots were pre-gated on live single cells using a viability dye (FVD-eFlour780).

FIG. 6—SupT1 transduced with retroviral vector encoding relevant antigens. A) Antigen expression on CD123, CD33 and CLL1 transduced SupT1 cells are depicted in blue, while red peaks correspond to relevant isotype controls. B) Mean antigen density (Per cell) density within each transduced SupT1 cells using Quantibrite™ beads by flow cytometry.

FIG. 7—Analysis of antigen density using Quantibrite™ beads by flow cytometry. A) Antigen expression on target cells stained with CD33, CD123 and CLL1 (blue) compared with the relevant isotype control (red). Mean antigen density (per cell) for each antigen was measured.

FIG. 8—An example for T cells separation from target cells by using anti-CD3 antibody on SupT1 NT for aCD33 CARs.

FIG. 9—Cytotoxicity assay using all aCD33 CARs on different AML cells in three donors. Cytotoxicity was measured at different time intervals depending on the cells (HL-60: 24 hr), (SUPT1, MOLM and THP1: 48 hr). 12783 (aCD19 FMC63 scFv) used as a negative control and 27983 (aCD33 scFv) used as a positive control. All data were normalized to the non-transduced control.

FIGS. 10—24 hour Cytotoxicity assay for all CD123-VHH-CAR T cell constructs. Each condition was tested with a minimum of 3 donors (n=3), with median indicated. Top row—SupT1 NT were used as target cells, middle row—SupT1 cells were engineered to express high levels of CD123, bottom row—AML derived KG1α were used as target cell line. All data were normalised to non-transduced T-cells as they only contribute to antigen independent cytotoxicity. CARs were compared by two-way paired t-test. * p<0.05, ** p<0.01, *** p<0.001.

FIGS. 11—48 hour Cytotoxicity assay for all CD123-VHH-CAR T cell constructs. Each condition was tested with a minimum of 3 donors (n=3), with median indicated. From left to right target cell lines SupT1 NT, THP1, and Molm14 were used as target cell lines. All data were normalised to non-transduced T-cells as they only contribute to antigen independent cytotoxicity.

FIGS. 12—24 hour Cytotoxicity assay for all CLL1-VHH-CAR T cell constructs. Each condition was tested with a minimum of 3 donors (n=3), with median indicated. Top row—SupT1 NT were used as target cells, bottom row—SupT1 cells were engineered to express high levels of CLL1. All data were normalised to non-transduced T-cells as they only contribute to antigen independent cytotoxicity.

FIGS. 13—48 hour Cytotoxicity assay for all CII1-VHH-CAR T cell constructs. Each condition was tested with a minimum of 3 donors (n=3), with median indicated. Top row—SupT1 NT were used as target cells, middle row—THP1 patient derived taget Is, bottom row—KG1α patient derived target cell line. All data were normalised to non-transduced T-cells as they only contribute to antigen independent cytotoxicity. CARs were compared by two-way paired t-test. * p<0.05, ** p<0.01, *** p<0.001, ns=no significance.

FIG. 14—IL-2 secretion of aCD33 CAR T cells co-culture with target cells in 1:1 E:T ratio in two donors (Donor 6 data not shown). The production of IL-2 was measured using engineered SupT1 cells express higher level of CD33 and AML derived cells.

FIG. 15—IFN-γ production of the aCD33 CAR T cells in target cells in 1:1 E:T ratio in two donors.

FIG. 16—Cytokine measurements after 24 and 48 hr cytotoxicity assay using CD123-VHH-CAR T cell constructs. Conducted at a 1:8 ratio for three donors (n=3). A) IL-2 measurements B) IFN-γ measurements. CARs were compared by two-way paired t-test. * p<0.05, ** p<0.01, *** p<0.001, ns =no significance.

FIG. 17—Cytokine measurements after 24 and 48hr cytotoxicity assay using CLL1-VHH-CAR T cell constructs. Conducted at a 1:8 ratio for three donors (n=3). A) IL-2 measurements B) IFN-γ measurements. CARs were compared by two-way paired t-test. * p<0.05, ** p<0.01, *** p<0.001, ns =no significance.

FIG. 18—Proliferation assay of each aCD33 CAR T cells on different cell lines. 12783 (aCD19 FMC63 scFv) used as a negative control and 27983 (aCD33 scFv) used as a positive control. Fold expansion was normalized to the non-transduced control. Assay was setup in 1:1 E:T ratio for 6 days. Proliferation assay was setup with 2 donors in HL-60 cells.

FIG. 19—4 Day proliferation assay for all CD123-VHH-CAR T cell constructs. Fold expansion was normalised to CD3+ non-transduced T cell condition. Each condition was tested with a minimum of 3 donors (n=3).

FIG. 20—4 Day proliferation assay for all CLL1-VHH-CAR T cell constructs. Fold expansion was normalised to CD3+non-transduced T cell condition. Each condition was tested with a minimum of 3 donors (n=3). CARs were compared by two-way paired t-test. * p<0.05, ** p<0.01, *** p<0.001, ns=no significance.

### SUMMARY OF ASPECTS OF THE INVENTION

In a first aspect, the present invention provides a CAR-expressing cell which targets multiple antigens associated with acute myeloid leukemia (AML). The cell targets three or four of the following antigens: CD33, CLL-1, CD123 and FLT3.

For example the cell may comprise: an anti-CD33 chimeric antigen receptor (CAR); an anti-CLL1 CAR; and an anti-CD123 CAR.

The cell may comprise: an anti-CD33 chimeric antigen receptor (CAR); an anti-CLL1 CAR; and an anti-FLT3 CAR.

The cell may comprise: an anti-CD33 chimeric antigen receptor (CAR); an anti-CLL1 CAR; and an anti-CD123 CAR; and an anti-FLT3 CAR.

One or more, or all, of the CAR(s) may comprise a domain antibody (dAb) antigen binding domain.

The cell may comprise one or more tandem chimeric antigen receptor(s) (tanCAR(s)). The or each TanCAR may comprise domain antibody (dAb) antigen binding domains.

An anti-CD33 CAR with a domain antibody (dAb) antigen binding domain may comprise the following complementarity determining regions:

(i)
CDR1 -
(SEQ ID No. 1)
GRTFSMHS;
CDR2 -
(SEQ ID No. 2)
VTWSGDTF;
CDR3 -
(SEQ ID No. 3)
KDDPYRPAYDY;
(ii)
CDR1 -
(SEQ ID No. 4)
GRTFSSYV;
CDR2 -
(SEQ ID No. 5)
ISWSGGST;
CDR3 -
(SEQ ID No. 6)
AAMELRGGSYNYASSRQYDY;
(iii)
CDR1 -
(SEQ ID No. 7)
EIAFSNFN;
CDR2 -
(SEQ ID No. 8)
ISSHGDTNY;
CDR3 -
(SEQ ID No. 9)
NANDPFLSVSDF;
(iv)
CDR1 -
(SEQ ID No. 10)
GSIFSINA;
CDR2 -
(SEQ ID No. 5)
ISWSGGST;
CDR3 -
(SEQ ID No. 11)
AAISGWGRSIRVGERYEYDY;
(v)
CDR1 -
(SEQ ID No. 12)
GRTSSSST;
CDR2 -
(SEQ ID No. 13)
ITLSGGST;
CDR3 -
(SEQ ID No. 14)
AARRWSNNRGGYDRAGYDY;
or
(vi)
CDR1 -
(SEQ ID No. 15)
GRTFSSYA;
CDR2 -
(SEQ ID No. 16)
ITWSGGST;
CDR3 -
(SEQ ID No. 17)
AMLLRGGLYDYTDYILYNY.

An anti-CD33 CAR with a domain antibody (dAb) antigen binding domain may comprise one of the sequences shown as SEQ ID No. 18, 19, 20, 21, 22 or 23.

An anti-CLL-1 CAR with a domain antibody (dAb) antigen binding domain may comprise the following complementarity determining regions:

(i)
CDR1 -
(SEQ ID No. 48)
GFTFGNHD;
CDR2 -
(SEQ ID No. 49)
IDSGGNVI;
CDR3 -
(SEQ ID No. 50)
ATDLDSGAESLESVY;
(ii)
CDR1 -
(SEQ ID No. 51)
GFAFGSAD;
CDR2 -
(SEQ ID No. 52)
IDSGGNTQ;
CDR3 -
(SEQ ID No. 53)
TDLDPTTDSLENVY;
(iii)
CDR1 -
(SEQ ID No. 54)
GRTFSAYF;
CDR2 -
(SEQ ID No. 55)
INWNGDSS;
CDR3 -
(SEQ ID No. 56)
AADTHGAVGLGSERLYDY;
(iv)
CDR1 -
(SEQ ID No. 57)
GIGVSSTG;
CDR2 -
(SEQ ID No. 58)
IDRDGTT;
CDR3 -
(SEQ ID No. 59)
TVVGDYY;
(v)
CDR1 -
(SEQ ID No. 60)
GFIFGNYD;
CDR2 -
(SEQ ID No. 61)
ISSGGNDI;
CDR3 -
(SEQ ID No. 62)
AADLDPGTDSLDNIH;
or
(vi)
CDR1 -
(SEQ ID No. 63)
GFTLDYYA;
CDR2 -
(SEQ ID No. 64)
ISSSDGST;
CDR3 -
(SEQ ID NO. 65)
AEAVYYAGVCVAMYDS.

A anti-CLL-1 CAR with a domain antibody (dAb) antigen binding domain may comprise one of the sequences shown as SEQ ID No. 66, 67, 68, 69, 70 or 71.

A anti-CD123 CAR with a domain antibody (dAb) antigen binding domain may comprise the following complementarity determining regions:

(i)
CDR1 -
(SEQ ID No. 24)
GRSINTYA;
CDR2 -
(SEQ ID No. 25)
INYNSRYT;
CDR3 -
(SEQ ID No. 26)
AATSYYPTDYDVASRVATWPS;
(ii)
CDR1 -
(SEQ ID No. 27)
GISLNA;
CDR2 -
(SEQ ID No. 28)
IKIGGVS;
CDR3 -
(SEQ ID No. 29)
NTYPPYLNGMDY;
(iii)
CDR1 -
(SEQ ID No. 30)
GRSFNTDA;
CDR2 -
(SEQ ID No. 31)
ISWDGTRT;
CDR3 -
(SEQ ID No. 32)
AAEPQKAWPIGTSAAGFRS;
(iv)
CDR1 -
(SEQ ID No. 33)
GSSISV;
CDR2 -
(SEQ ID No. 34)
ISWSDGNT;
CDR3 -
(SEQ ID No. 35)
AVEPRGWPKGHRY;
(v)
CDR1 -
(SEQ ID No. 36)
GSSFSINV;
CDR2 -
(SEQ ID No. 37)
ISWSDGST;
CDR3 -
(SEQ ID No. 38)
AVEPRGWPKGHRY;
or
(vi)
CDR1 -
(SEQ ID No. 39)
GSIFRINA;
CDR2 -
(SEQ ID No. 40)
VNWIGGTT;
CDR3 -
(SEQ ID No. 41)
SATDKGGSSRY.

A anti-CD123 CAR with a domain antibody (dAb) antigen binding domain may comprise one of the sequences shown as SEQ ID No. 42, 43, 44, 45, 46 or 47.

An anti-FLT3 CAR with a domain antibody (dAb) antigen binding domain may comprise the following complementarity determining regions:

(i)
CDR1 -
(SEQ ID No. 72)
GIFKTNY;
CDR2 -
(SEQ ID No. 73)
FTNDGST;
CDR3 -
(SEQ ID No. 74)
YGLGH;
(ii)
CDR1 -
(SEQ ID No. 75)
GTISSIRY;
CDR2 -
(SEQ ID No. 76)
ITSSGNT;
CDR3 -
(SEQ ID No. 77)
YTMGY;
(iii)
CDR1 -
(SEQ ID No. 78)
GIFSTNY;
CDR2 -
(SEQ ID No. 79)
FTNDGGT;
CDR3 -
(SEQ ID No. 80)
CGLGH;
(iv)
CDR1 -
(SEQ ID No. 81)
GSISSIRY;
CDR2 -
(SEQ ID No. 82)
ITSSGST;
CDR3 -
(SEQ ID No. 83)
YTMGY;
or
(v)
CDR1 -
(SEQ ID No. 84)
GIFSTNH;
CDR2 -
(SEQ ID No. 85)
FTNDGST;
CDR3 -
(SEQ ID NO. 86)
YGLGH.

An anti-FLT3 CAR with a domain antibody (dAb) antigen binding domain may comprise one of the sequences shown as SEQ ID No. 87, 88, 89, 90 or 91.

In a second aspect, the present invention provides a nucleic acid construct which encodes a plurality of CARs. The nucleic acid construct may encode CARs against three or all four of the following antigens: CD33, CLL-1, CD123 and FLT3. For example, the nucleic acid construct may encode: an anti-CD33 chimeric antigen receptor (CAR); an anti-CLL1 CAR; and an anti-CD123 CAR.

The nucleic acid construct may encode: an anti-CD33 chimeric antigen receptor (CAR); an anti-CLL1 CAR; and an anti-FLT3 CAR.

The nucleic acid construct may encode: an anti-CD33 chimeric antigen receptor (CAR); an anti-CLL1 CAR; and an anti-CD123 CAR; and an anti-FLT3 CAR.

In a third aspect, the present invention provides a method for making a cell according to the first aspect of the invention which comprises the step of transducing or transfecting a cell with a nucleic acid construct according to the second aspect of the invention.

In a fourth aspect, the present invention provides a vector comprising a nucleic acid construct according to the second aspect of the invention.

In a fifth aspect, there is provided a kit of vectors, which comprises a plurality of vectors, each encoding a CAR against a target antigen. The kit may comprise vectors encoding CARs against three, or all four of the following target antigens: CD33, CLL-1, CD123 and FLT3. For example, the kit may comprise:
(i) a first vector which comprises a nucleic acid sequence encoding a chimeric antigen receptor (CAR) which binds CD33;
(ii) a second vector which comprises a nucleic acid sequence encoding a CAR which binds CLL1; and
(iii) a third vector which comprises a nucleic acid sequence encoding a CAR which binds CD123.

The kit may comprise:
(i) a first vector which comprises a nucleic acid sequence encoding a chimeric antigen receptor (CAR) which binds CD33;
(ii) a second vector which comprises a nucleic acid sequence encoding a CAR which binds CLL1; and
(iii) a third vector which comprises a nucleic acid sequence encoding a CAR which binds FLT3.

The kit may comprise:
(i) a first vector which comprises a nucleic acid sequence encoding a chimeric antigen receptor (CAR) which binds CD33;
(ii) a second vector which comprises a nucleic acid sequence encoding a CAR which binds CLL1;
(iii) a third vector which comprises a nucleic acid sequence encoding a CAR which binds CD123; and
(iv) a fourth vector which comprises a nucleic acid sequence encoding a CAR which binds FLT3.

In a sixth aspect there is provided a pharmaceutical composition which comprises a plurality of cells according to the first aspect of the invention, together with a pharmaceutically acceptable carrier, diluent or excipient.

In a seventh aspect, there is provided a method for treating cancer which comprises the step of administering a pharmaceutical composition according to the sixth aspect of the invention to a subject.

The cancer may be acute myeloid leukemia (AML).

The method may also involve the step of subsequently administering an allogeneic transplant to the subject.

In an eighth aspect, there is provided a pharmaceutical composition according to the sixth aspect of the invention for use in treating cancer.

In a ninth aspect, there is provided the use of a cell according to the first aspect of the invention in the manufacture of a pharmaceutical composition for treating cancer.

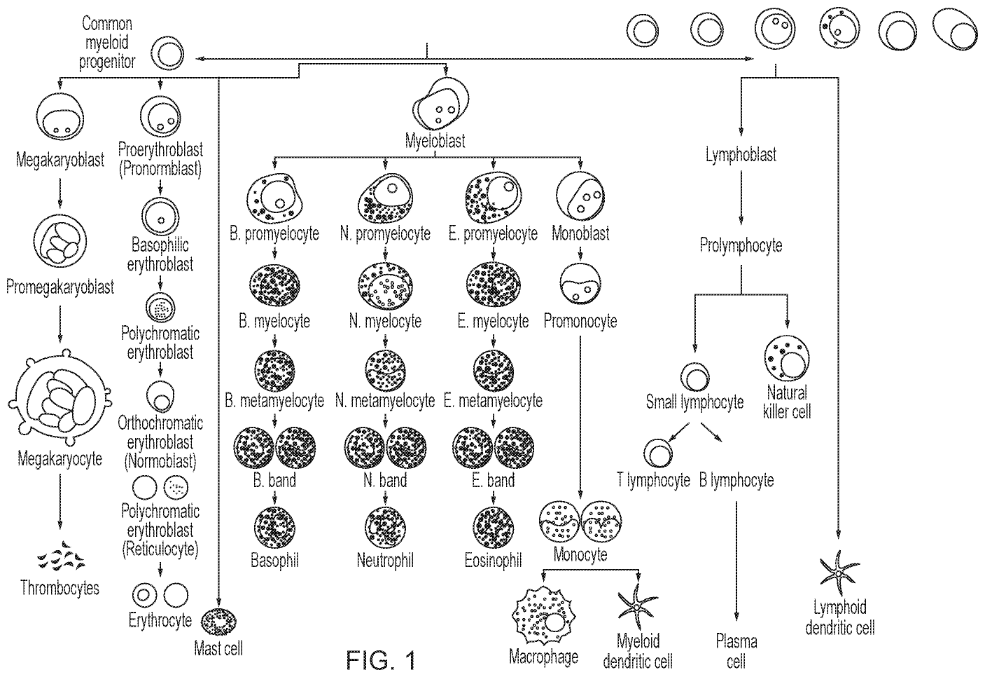

AML blast phenotype is much more heterogenous than that of acute lymphoblastic leukemia (ALL) blasts. Myelopoiesis is driven by stem cells which stochastically and in response to cues either replenish their compartment or differentiate. Akin to normal myeloid stem cells, AML stem cells propagate or differentiate to cause bone-marrow replacement with a range of cells at different differentiation states. AML stem cells can occur along the range of myelopoiesis and consequently have different surface antigen profile. Further, in a given patient, there may be stem cell nexi or a hierarchy of different stem cells at different points in ontogeny all contributing to the disease burden (FIG. 1).

In order to treat the maximum number of patients, the present inventors have found that AML targeting by chimeric antigen receptors requires targeting of multiple antigens simultaneously along the myeloid lineage.

The OR gates of the present invention provide an advantage over the current phase I clinical trials of CAR T-cell immunotherapy for patients with relapsed/refractory AML shown in Table 1 above. Targeting a single antigen may fail to eliminate the disease-relevant stem cell compartment. Targeting multiple myeloid antigens simultaneously, however, means that the treatment is universal across AMLs. Immunotherapy using CAR-T cells targeting multiple antigens eradicates the disease stem cell compartment irrespective of the number and position of stem cell compartments. Targeting multiple antigens also reduces the likelihood of escape by antigen down-regulation.

### FURTHER ASPECTS

The present invention also provides a cell composition comprising CAR-expressing cells expressing multiple CARs.

The composition of cells may express: an anti-CD33 chimeric antigen receptor (CAR); an anti-CLL1 CAR; and an anti-CD123 CAR.

The composition of cells may express: an anti-CD33 chimeric antigen receptor (CAR); an anti-CLL1 CAR; and an anti-FLT3 CAR.

The composition of cells may express: an anti-CD33 chimeric antigen receptor (CAR); an anti-CLL1 CAR; and an anti-CD123 CAR; and an anti-FLT3 CAR.

The cells of the composition may each express one CAR type. For example, the composition may comprise a mixture of one of the following:
CD33 CAR-expressing cells, CLL-1 CAR-expressing cells and CD123 CAR-expressing cells;
CD33 CAR-expressing cells, CLL-1 CAR-expressing cells and FLT3 CAR-expressing cells; or
CD33 CAR-expressing cells, CLL-1 CAR-expressing cells, CD123 CAR-expressing cells and FLT3 CAR-expressing cells.

Alternatively, at least some of the cells of the composition may express more than one CAR, for example, the composition may comprise a combination of:
cells expressing CD33 CAR/CLL-1 CAR OR gate and cells expressing CD123 CAR;
cells expressing CD33 CAR/CD123 CAR OR gate and cells expressing CLL-1 CAR;
cells expressing CD123 CAR/CLL-1 CAR OR gate and cells expressing CD33 CAR;
cells expressing CD33 CAR/CLL-1 CAR OR gate and cells expressing FLT3 CAR;
cells expressing CD33 CAR/FLT3 CAR OR gate and cells expressing CLL-1 CAR;
cells expressing FLT3 CAR/CLL-1 CAR OR gate and cells expressing CD33 CAR;
cells expressing CD33 CAR/CLL-1 CAR OR gate and cells expressing CD123 CAR/FLT3 CAR OR gate;
cells expressing CD123 CAR/CLL-1 CAR OR gate and cells expressing CD33 CAR/FLT3 CAR OR gate; or
cells expressing CD33 CAR/CD123 CAR OR gate and cells expressing CLL-1 CAR/FLT3 CAR OR gate.

At least some of the cells of the composition may express a tanCAR as described below. For example, the composition may comprise a combination of:
cells expressing CD33/CLL-1 tanCAR and cells expressing CD123 CAR;
cells expressing CD33/CD123 tanCAR and cells expressing CLL-1 CAR;
cells expressing CD123/CLL-1 tanCAR and cells expressing CD33 CAR;
cells expressing CD33/CLL-1 tanCAR and cells expressing FLT3 CAR;
cells expressing CD33/FLT3 tanCAR and cells expressing CLL-1 CAR;
cells expressing FLT3/CLL-1 tanCAR and cells expressing CD33 CAR;
cells expressing CD33/CLL-1 tanCAR and cells expressing CD123/FLT3 tanCAR;
cells expressing CD123/CLL-1 tanCAR and cells expressing CD33/FLT3 tanCAR; or
cells expressing CD33/CD123 tanCAR and cells expressing CLL-1/FLT3 tanCAR.

The nucleic acid sequences, nucleic acid constructs, vectors and kits of vectors and methods described below may be used to make the cells of the cell composition of this aspect of the invention.

The cell composition may be used in a method for treating a disease, as described below.

Yet further aspects of the invention are summarised in the following numbered paragraphs:
A1. A domain antibody (dAb) which binds CD33 and comprises the following complementarity determining regions (CDRs):

(i)
CDR1 -
(SEQ ID No. 1)
GRTFSMHS;
CDR2 -
(SEQ ID No. 2)
VTWSGDTF;
CDR3 -
(SEQ ID No. 3)
KDDPYRPAYDY;
(ii)
CDR1 -
(SEQ ID No. 4)
GRTFSSYV;
CDR2 -
(SEQ ID No. 5)
SWSGGST;
CDR3 -
(SEQ ID No. 6)
AAMELRGGSYNYASSRQYDY;
(iii)
CDR1 -
(SEQ ID No. 7)
EIAFSNFN;
CDR2 -
(SEQ ID No. 8)
ISSHGDTNY;
CDR3 -
(SEQ ID No. 9)
NANDPFLSVSDF;
(iv)
CDR1 -
(SEQ ID No. 10)
GSIFSINA;
CDR2 -
(SEQ ID No. 5)
ISWSGGST;
CDR3 -
(SEQ ID No. 11)
AAISGWGRSIRVGERYEYDY;
(v)
CDR1 -
(SEQ ID No. 12)
GRTSSSST;
CDR2 -
(SEQ ID No. 13)
ITLSGGST;
CDR3 -
(SEQ ID No. 14)
AARRWSNNRGGYDRAGYDY;
or
(vi)
CDR1 -
(SEQ ID No. 15)
GRTFSSYA;
CDR2 -
(SEQ ID No. 16)
ITWSGGST;
CDR3 -
(SEQ ID NO. 17)
AMLLRGGLYDYTDYILYNY.

A2. A dAb according to paragraph A1 which comprises one of the sequences shown as SEQ ID No. 18, 19, 20, 21, 22 or 23.
A3. A chimeric antigen receptor (CAR) which has an antigen binding domain comprising a dAb according to paragraph A1 or A2.
A4. A nucleic acid sequence encoding a dAb according to paragraph A1 or A2 or a CAR according to paragraph A3.
A5. A vector comprising a nucleic acid sequence according to paragraph A4.
A6. A cell which expresses a CAR according to paragraph A3.
A7. A method for making a cell according to paragraph A6 which comprises the step of transducing or transfecting a cell with a vector according to paragraph A5.
A8. A pharmaceutical composition comprising a plurality of cells according to paragraph A6.
A9. A method for treating cancer which comprises the step of administering a pharmaceutical composition according to paragraph A8 to a subject.
A10. A method according to paragraph A9, wherein the cancer is acute myeloid leukemia (AML).
A11. A pharmaceutical composition according to paragraph A8 for use in treating cancer.
A12. The use of a cell according to paragraph A6 in the manufacture of a pharmaceutical composition for treating cancer.
B1. A domain antibody (dAb) which binds CD123 and comprises the following complementarity determining regions (CDRs):

(i)
CDR1 -
(SEQ ID No. 24)
GRSINTYA;
CDR2 -
(SEQ ID No. 25)
INYNSRYT;
CDR3 -
(SEQ ID No. 26)
AATSYYPTDYDVASRVATWPS;
(ii)
CDR1 -
(SEQ ID No. 27)
GISLNA;
CDR2 -
(SEQ ID No. 28)
IKIGGVS;
CDR3 -
(SEQ ID No. 29)
NTYPPYLNGMDY;
(iii)
CDR1 -
(SEQ ID No. 30)
GRSFNTDA;
CDR2 -
(SEQ ID No. 31)
ISWDGTRT;
CDR3 -
(SEQ ID No. 32)
AAEPQKAWPIGTSAAGFRS;
(iv)
CDR1 -
(SEQ ID No. 33)
GSSISV;
CDR2 -
(SEQ ID No. 34)
ISWSDGNT;
CDR3 -
(SEQ ID No. 35)
AVEPRGWPKGHRY;
(v)
CDR1 -
(SEQ ID No. 36)
GSSFSINV;
CDR2 -
(SEQ ID No. 37)
ISWSDGST;
CDR3 -
(SEQ ID No. 38)
AVEPRGWPKGHRY;
or
(vi)
CDR1 -
(SEQ ID No. 39)
GSIFRINA;
CDR2 -
(SEQ ID No. 40)
VNWIGGTT;
CDR3 -
(SEQ ID NO. 41)
SATDKGGSSRY.

B2. A dAb according to paragraph B1 which comprises one of the sequences shown as SEQ ID No: 42, 43, 44, 45, 46 or 47.
B3. A chimeric antigen receptor (CAR) which has an antigen binding domain comprising a dAb according to paragraph B1 or B2.
B4. A nucleic acid sequence encoding a dAb according to paragraph B1 or B2 or a CAR according to paragraph B3.
B5. A vector comprising a nucleic acid sequence according to paragraph B4.
B6. A cell which expresses a CAR according to paragraph B3.
B7. A method for making a cell according to paragraph B6 which comprises the step of transducing or transfecting a cell with a vector according to paragraph B5.
B8. A pharmaceutical composition comprising a plurality of cells according to paragraph B6.
B9. A method for treating cancer which comprises the step of administering a pharmaceutical composition according to paragraph B8 to a subject.
B10. A method according to paragraph B9, wherein the cancer is acute myeloid leukemia (AML).
B11. A pharmaceutical composition according to paragraph B8 for use in treating cancer.
B12. The use of a cell according to paragraph B6 in the manufacture of a pharmaceutical composition for treating cancer.

The present invention provides a domain antibody (dAb) which binds FLT3 and comprises the following complementarity determining regions:
C1. A domain antibody (dAb) which binds FLT3 and comprises the following complementarity determining regions (CDRs):

(i)
CDR1 -
(SEQ ID No. 72)
GIFKTNY;
CDR2 -
(SEQ ID No. 73)
FTNDGST;
CDR3 -
(SEQ ID No. 74)
YGLGH;
(ii)
CDR1 -
(SEQ ID No. 75)
GTISSIRY;
CDR2 -
(SEQ ID No. 76)
ITSSGNT;
CDR3 -
(SEQ ID No. 77)
YTMGY;
(iii)
CDR1 -
(SEQ ID No. 78)
GIFSTNY;
CDR2 -
(SEQ ID No. 79)
FTNDGGT;
CDR3 -
(SEQ ID No. 80)
CGLGH;
(iv)
CDR1 -
(SEQ ID No. 81)
GSISSIRY;
CDR2 -
(SEQ ID No. 82)
ITSSGST;
CDR3 -
(SEQ ID No. 83)
YTMGY;
or
(v)
CDR1 -
(SEQ ID No. 84)
GIFSTNH;
CDR2 -
(SEQ ID No. 85)
FTNDGST;
CDR3 -
(SEQ ID No. 86)
YGLGH.

C2. A dAb according to paragraph C1 which comprises one of the sequences shown as SEQ ID No. 87, 88, 89, 90 or 91.
C3. A chimeric antigen receptor (CAR) which has an antigen binding domain comprising a dAb according to paragraph C1 or C2.
C4. A nucleic acid sequence encoding a dAb according to paragraph C1 or C2 or a CAR according to paragraph C3.
C5. A vector comprising a nucleic acid sequence according to paragraph C4.
C6. A cell which expresses a CAR according to paragraph C3.
C7. A method for making a cell according to paragraph C6 which comprises the step of transducing or transfecting a cell with a vector according to paragraph C5.
C8. A pharmaceutical composition comprising a plurality of cells according to paragraph C6.
C9. A method for treating cancer which comprises the step of administering a pharmaceutical composition according to paragraph C8 to a subject.
C10. A method according to paragraph C9, wherein the cancer is acute myeloid leukemia (AML).
C11. A pharmaceutical composition according to paragraph C8 for use in treating cancer.
C12. The use of a cell according to paragraph C6 in the manufacture of a pharmaceutical composition for treating cancer.
D1. A domain antibody (dAb) which binds CLL1 and comprises the following complementarity determining regions (CDRs):

(i)
CDR1 -
(SEQ ID No. 48)
GFTFGNHD;
CDR2 -
(SEQ ID No. 49)
IDSGGNVI;
CDR3 -
(SEQ ID No. 50)
ATDLDSGAESLESVY;
(ii)
CDR1 -
(SEQ ID No. 51)
GFAFGSAD;
CDR2 -
(SEQ ID No. 52)
IDSGGNTQ;
CDR3 -
(SEQ ID No. 53)
TDLDPTTDSLENVY;
(iii)
CDR1 -
(SEQ ID No. 54)
GRTFSAYF;
CDR2 -
(SEQ ID No. 55)
INWNGDSS;
CDR3 -
(SEQ ID No. 56)
AADTHGAVGLGSERLYDY;
(iv)
CDR1 -
(SEQ ID No. 57)
GIGVSSTG;
CDR2 -
(SEQ ID No. 58)
IDRDGTT;
CDR3 -
(SEQ ID No. 59)
TVVGDYY;
(v)
CDR1 -
(SEQ ID No. 60)
GFIFGNYD;
CDR2 -
(SEQ ID No. 61)
ISSGGNDI;
CDR3 -
(SEQ ID No. 62)
AADLDPGTDSLDNIH;
or
(vi)CDR1 -
(SEQ ID No. 63)
GFTLDYYA;
CDR2 -
(SEQ ID No. 64)
ISSSDGST;
CDR3 -
(SEQ ID NO. 65)
AEAVYYAGVCVAMYDS.

D2. A dAb according to paragraph D1 which comprises one of the sequences shown as SEQ ID No. SEQ ID No. 66, 67, 68, 69, 70 or 71.
D3. A chimeric antigen receptor (CAR) which has an antigen binding domain comprising a dAb according to paragraph D1 or D2.
D4. A nucleic acid sequence encoding a dAb according to paragraph D1 or D2 or a CAR according to paragraph D3.
D5. A vector comprising a nucleic acid sequence according to paragraph D4.
D6. A cell which expresses a CAR according to paragraph D3.
D7. A method for making a cell according to paragraph D6 which comprises the step of transducing or transfecting a cell with a vector according to paragraph D5.
D8. A pharmaceutical composition comprising a plurality of cells according to paragraph D6.
D9. A method for treating cancer which comprises the step of administering a pharmaceutical composition according to paragraph D8 to a subject.
D10. A method according to paragraph D9, wherein the cancer is acute myeloid leukemia (AML).
D11. A pharmaceutical composition according to paragraph D8 for use in treating cancer.
D12. The use of a cell according to paragraph D6 in the manufacture of a pharmaceutical composition for treating cancer.

### DETAILED DESCRIPTION

### CHIMERIC ANTIGEN RECEPTORS

The present invention relates to a cell which expresses a plurality of chimeric antigen receptors at the cell surface.

A classical chimeric antigen receptor (CAR) is a chimeric type I trans-membrane protein which connects an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FccR1 or CD3. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3 results in second generation receptors which can transmit an activating and co-stimulatory signal simultaneously after antigen recognition. The co-stimulatory domain most commonly used is that of CD28. This supplies the most potent co-stimulatory signal -namely immunological signal 2, which triggers T-cell proliferation. Some receptors have also been described which include TNF receptor family endodomains, such as the closely related OX40 and 41 BB which transmit survival signals. Even more potent third generation CARs have now been described which have endodomains capable of transmitting activation, proliferation and survival signals.

When a CAR binds the target-antigen, this results in the transmission of an activating signal to the T-cell on which it is expressed. Thus, the CAR directs the specificity and cytotoxicity of the T cell towards tumour cells expressing the targeted antigen.

CARs typically comprise: (i) an antigen-binding domain; (ii) a spacer; (iii) a transmembrane domain; and (iii) an intracellular domain which comprises or associates with a signalling domain.

A CAR may have the general structure:

Antigen binding domain—spacer domain—transmembrane domain—intracellular signaling domain (endodomain).

### ANTIGEN BINDING DOMAIN

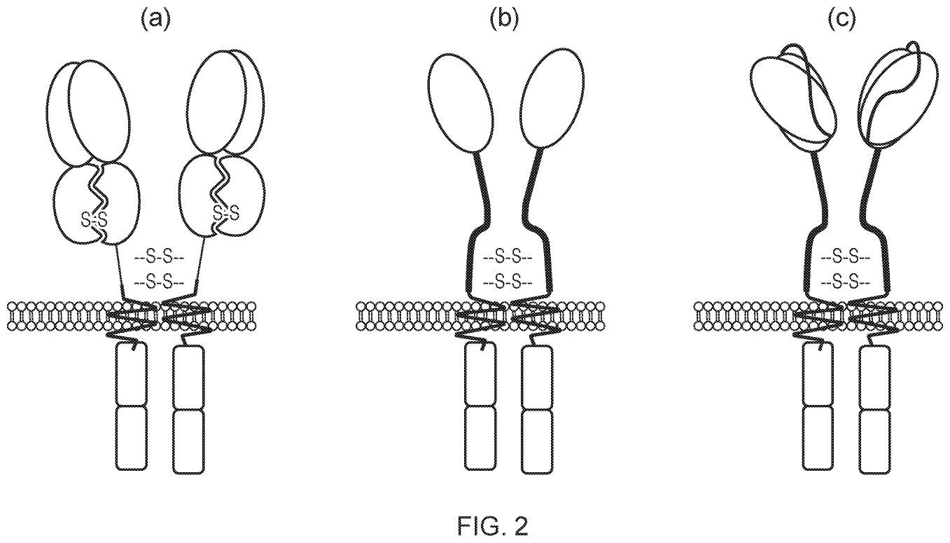

The antigen binding domain is the portion of the chimeric receptor which recognizes antigen. In a classical CAR, the antigen-binding domain comprises a single-chain variable fragment (scFv) derived from a monoclonal antibody (see FIG. 2c). CARs have also been produced with domain antibody (dAb) or VHH antigen binding domains (see FIG. 2b); or in a Fab CAR format (FIG. 2a). A FabCAR comprises two chains: one having an antibody-like light chain variable region (VL) and constant region (CL); and one having a heavy chain variable region (VH) and constant region (CH). One chain also comprises a transmembrane domain and an intracellular signalling domain. Association between the CL and CH causes assembly of the receptor.

The antigen binding domain(s) of the CAR may be a single domain binder, also known as a “dAb”, “VHH”, “domain antibody” or “nanobody”.

A conventional IgG molecule is comprised of two heavy and two light chains. Heavy chains comprise three constant domains and one variable domain (VH); light chains comprise one constant domain and one variable domain (VL). The naturally functional antigen binding unit is formed by noncovalent association of the VH and the VL domain. This association is mediated by hydrophobic framework regions.

A single-domain antibody is an antibody fragment consisting of a single monomeric variable antibody domain. The first single-domain antibodies were engineered from heavy-chain antibodies found in camelids which lack the light chain and the CH1 domain of a classical antibody. These heavy chain antibodies comprise a single, antigen binding domain, the VHH domain. Cartilaginous fishes also have heavy-chain antibodies (IgNAR, ‘immunoglobulin new antigen receptor’), from which single-domain antibodies called VNAR fragments can be obtained. An alternative approach is to split the dimeric variable domains from common immunoglobulin G (IgG) from humans or mice into monomers. Although most research into single-domain antibodies is currently based on heavy chain variable domains, Nanobodies derived from light chains have also been shown to bind specifically to target epitopes.

A single-domain antibody can be obtained by immunization of dromedaries, camels, llamas, alpacas or sharks with the desired antigen and subsequent isolation of the mRNA coding for heavy-chain antibodies. By reverse transcription and polymerase chain reaction, a gene library of single-domain antibodies may be produced. Screening techniques like phage display and ribosome display help to identify the clones binding the antigen. Alternatively, single-domain antibodies can be made from common murine or human IgG with four chains.

The present invention relates to the targeting of multiple antigens. This can be achieved by a number of approaches, including OR gates and tanCARs (as described in more detail below). Domain antibody antigen binding domains are particularly suited for such approaches because they are discrete and do not have a tendency to concatenate. They are also less complex, meaning that expression and folding are less likely to be compromised and that the sequence coding for such CAR/tanCARs requires less space on a viral vector genome.

### TANCARs

The cell of the present invention may comprise a TanCAR.

Bispecific CARs known as tandem CARs or TanCARs have been developed to target two or more cancer specific markers simultaneously. In a TanCAR, the extracellular domain comprises two antigen binding specificities in tandem, joined by a linker. The two binding specificities (scFvs) are thus both linked to a single transmembrane portion: one scFv being juxtaposed to the membrane and the other being in a distal position. When a TanCAR binds either or both of the target antigens, this results in the transmission of an activating signal to the cell it is expressed on.

Grada et al (2013, Mol Ther Nucleic Acids 2:e105) describes a TanCAR which includes a CD19-specific scFv, followed by a Gly-Ser linker and then a HER2-specific scFv. The HER2-scFv was in the juxta-membrane position, and the CD19-scFv in the distal position. The TanCAR was shown to induce distinct T cell reactivity against each of the two tumour restricted antigens. This arrangement was chosen because the respective lengths of HER2 (632 aa/125Å) and CD19 (280aa, 65Å) lends itself to that spatial arrangement. It was also known that the HER2 scFv bound the distal-most 4 loops of HER2.

The cell of the invention may comprises a TanCAR comprising two antigen binding specificities in tandem. The tanCAR may bind one of the following pairs of antigens: CD33 and CD123; CD33 and CLL-1, CD33 and FLT-3; CD123 and CLL-1; CD123 and FLT-3; CII1 and FLT-3.

In each of these antigen pairs, the antigen binding domains may be in either order in the molecule. For example, for the target antigen pair CD33 and CD123, the CD33-binding antigen binding domain may be juxtaposed to the membrane and the CD123-binding antigen binding domain may be distal to the membrane; or the CD123-binding antigen binding domain may be juxtaposed to the membrane and the CD33-binding antigen binding domain may be distal to the membrane.

The cell of the present invention may comprise a combination of a tanCAR and a CAR which a single antigen specificity such as an scFv-CAR or a dAb CAR. In this respect, the cell has three antigen specificities: two for the TanCAR and one for the scFv or dAb CAR. The cell may, for example comprise one of the combinations shown in Table 2.

TABLE 2

| Tan CAR specificity | scFv/dAb CAR specificity |
|---|---|
| CD33/CLL-1 | CD123 |
| CD33/CD123 | CLL-1 |
| CLL-1/CD123 | CD33 |
| CD33/CLL-1 | FLT3 |
| CD33/FLT3 | CLL1 |
| CLL-1/FLT3 | CD33 |

The cell of the present invention may comprise two tanCARs. For example, the cell may comprise dual tanCARs as shown in Table 3.

TABLE 3

| TanCAR1 specificity | TanCAR2 specificity |
|---|---|
| CD33/CLL-1 | CD123/FLT3 |
| CD33/CD123 | CLL-1/FLT3 |
| CD33/FLT-1 | CLL-1/CD123 |

### OR GATES

“Logic Gate” CAR combinations are described in WO2015/075469, WO2015/075470 and WO2015/075470. A CAR logic gate is a CAR combination which, when expressed by a cell, such as a T cell, is capable of detecting a particular pattern of expression of at least two target antigens. If the at least two target antigens are arbitrarily denoted as antigen A and antigen B, the three possible options are as follows:
“OR GATE”—T cell triggers when either antigen A or antigen B is present on the target cell
“AND GATE”—T cell triggers only when both antigens A and B are present on the target cell
“AND NOT GATE”—T cell triggers if antigen A is present alone on the target cell, but not if both antigens A and B are present on the target cell

Engineered T cells expressing these CAR combinations can be tailored to be exquisitely specific for cancer cells, based on their particular expression (or lack of expression) of two or more markers.

An “OR Gate” comprises two or more CARs each directed to a distinct target antigen expressed by a target cell. The advantage of an OR gate is that the effective targetable antigen is increased on the target cell, as it is effectively antigen A+antigen B. This is especially important for antigens expressed at variable or low density on the target cell, as the level of a single antigen may be below the threshold needed for effective targeting by a CAR-T cell. Also, it avoids the phenomenon of antigen escape. For example, some lymphomas and leukemias become CD19 negative after CD19 targeting: using an OR gate which targets CD19 in combination with another antigen provides a “back-up” antigen, should this occur.

The cell of the present invention may express a triple OR gate comprising three CARs. For example, the cell may express:
a CAR which binds CD33, a CAR which binds CLL-1; and a CAR which binds CD123 or
a CAR which binds CD33, a CAR which binds CLL-1; and a CAR which binds FLT3.
   The cell of the present invention may express a quadruple OR gate comprising four CARs. For example, the cell may express: a CAR which binds CD33, a CAR which binds CLL-1; a CAR which binds CD123; and a CAR which binds FLT3.

In the triple and quadruple OR gates of the invention, one of more CAR(s) may be a dAb CAR. In particular, all of the CARs of the cell may be dAb CARs.

### TARGET ANTIGEN

The, or one of the, antigen binding domain(s) of the CAR may specifically bind one of the following target antigens: CD33, CD123, CLL-1 and FLT-3

### CD33

CD33 is a myeloid differentiation antigen which is displayed on some normal B-cells and activated T- and natural killer cells but is not expressed on pluripotent hematopoietic stem cells or outside the hematopoietic system. It is found on at least a subset of blasts in nearly all acute myeloid leukemias (AMLs). With an average of 104 molecules/leukemic cell, CD33 is not highly abundant but levels vary considerably across individual patients.

The extracellular portion of CD33 contains two immunoglobulin domains and the intracellular portion contains innumoreceptor tyrosine-based inhibitory motifs (ITIMs). The amino acid sequence of human CD33 is available from Uniprot Accession number P20138.

Several commercially available antibodies against CD33 are known, such as WM-53, P67.6, HIM3-4 (Thermofisher).

The present invention provides a domain antibody (dAb) which binds CD33 and comprises the following complementarity determining regions:

(i)
CDR1 -
(SEQ ID No. 1)
GRTFSMHS;
CDR2 -
(SEQ ID No. 2)
VTWSGDTF;
CDR3 -
(SEQ ID No. 3)
KDDPYRPAYDY;
(ii)
CDR1 -
(SEQ ID No. 4)
GRTFSSYV;
CDR2 -
(SEQ ID No. 5)
ISWSGGST;
CDR3 -
(SEQ ID No. 6)
AAMELRGGSYNYASSRQYDY;
(iii)
CDR1 -
(SEQ ID No. 7)
EIAFSNFN;
CDR2 -
(SEQ ID No. 8)
ISSHGDTNY;
CDR3 -
(SEQ ID No. 9)
NANDPFLSVSDF;
(iv)
CDR1 -
(SEQ ID No. 10)
GSIFSINA;
CDR2 -
(SEQ ID No. 5)
ISWSGGST;
CDR3 -
(SEQ ID No. 11)
AAISGWGRSIRVGERYEYDY;
(v)
CDR1 -
(SEQ ID No. 12)
GRTSSSST;
CDR2 -
(SEQ ID No. 13)
ITLSGGST;
CDR3 -
(SEQ ID No. 14)
AARRWSNNRGGYDRAGYDY;
or
(vi)
CDR1 -
(SEQ ID No. 15)
GRTFSSYA;
CDR2 -
(SEQ ID No. 16)
ITWSGGST;
CDR3 -
(SEQ ID NO. 17)
AMLLRGGLYDYTDYILYNY.

The anti-CD33 dAb may comprise one of the sequences shown as SEQ ID No. 18, 19, 20, 21, 22 or 23.

(CD33 dAb P1.E4-44738)
SEQ ID No. 18
QVQLESGGGLVQAGGSLRLSCAASGRTFSMHSMGWFRQAPGKEREFVAA
VTWSGDTFAYADFVKGRFTISRGIAKNTLYLQMNSLKPEDTAVYYCAAK
DDPYRPAYDYWGQGTQVTVSS
(CD33 dAb P1.H3-44739)
SEQ ID No. 19
QVQLQESGGGLVQAGGSLRLSCAASGRTFSSYVMGWFRQAPGKEREFVA
AISWSGGSTYYADSVKGRFTISRDNAKNTLYLQMNSLKPEDTAVYYCAA
MELRGGSYNYASSRQYDYWGQGTQVTVSS
(CD33 dAb P1.G8-44742)
SEQ ID No. 20
QVQLQESGGGLVQTGGSLTLSCAASEIAFSNFNMGWYRQGSGKQRTLVA
QISSHGDTNYLDSMKGRFTISRDNNKKTVYLQMNALKPEDTAVYYCNAN
DPFLSVSDFWGQGTQVTVSS
(CD33 dAb P2.A7-46173)
SEQ ID No. 21
QVQLQQSGGGLVQAGGSLRLSCAASGSIFSINAMGWFRQAPGKEREFVA
AISWSGGSTYYADFVKGRFTISRDNAKNTVYLQMNSLKPEDTAIYYCAA
ISGWGRSIRVGERYEYDYWGQGTQVTVSS
(CD33 dAb P2.B12-46174)
SEQ ID No. 22
QVQLQESGGGLVQAGGSLRLSCAASGRTSSSSTMAWFRQAPGKEREFVA
AITLSGGSTHYADSAKGRFTISRESAKNTVYLQMNSLKPEDTADYYCAA
RRWSNNRGGYDRAGYDYWGQGTQVTVSS
(CD33 dAb P2.F2-46176)
SEQ ID No. 23
QVQLQESGGGLVQAGGSLRLSCAASGRTFSSYAMGWFRQAPGKEREFVA
AITWSGGSTYYADSVKGRFTISRDNAKNTLYLQMNSLKPEDTAVYYCAA
MLLRGGLYDYTDYILYNYWGQGTQVTVSS

The present invention also provides:
a CAR comprising such a CD33 dAb as antigen binding domain;
a nucleic acid sequence encoding such a dAb or CAR.

### CD123

CD123 is the transmembrane a subunit of the interleukin-3 receptor (IL-3Ra), which together with CD131 forms a high-affinity IL-3R. Upon binding of IL-3, IL-3R promotes cell proliferation and survival. CD123 is normally expressed at high levels on plasmacytoid dendritic cells, and basophils. It is expressed at low levels on monocytes, eosinophils, and myeloid dendritic cells. The amino acid sequence of human CD123 is available from NCBI Reference Sequence: NP_002174.1.

Several commercially available antibodies against CD123 are known, such as 6H6 and 5B11 (ThermoFisher).

The present invention provides a domain antibody (dAb) which binds CD123 and comprises the following complementarity determining regions:

(i)
CDR1
(SEQ ID No. 24)
GRSINTYA;
CDR2
(SEQ ID No. 25)
INYNSRYT;
CDR3
(SEQ ID No. 26)
AATSYYPTDYDVASRVATWPS;
(ii)
CDR1
(SEQ ID No. 27)
GISLNA;
CDR2
(SEQ ID No. 28)
IKIGGVS;
CDR3
(SEQ ID No. 29)
NTYPPYLNGMDY;
(iii)
CDR1
(SEQ ID No. 30)
GRSFNTDA;
CDR2
(SEQ ID No. 31)
ISWDGTRT;
CDR3
(SEQ ID No. 32)
AAEPQKAWPIGTSAAGFRS;
(iv)
CDR1
(SEQ ID No. 33)
GSSISV;
CDR2
(SEQ ID No. 34)
ISWSDGNT;
CDR3
(SEQ ID No. 35)
AVEPRGWPKGHRY;
(v)
CDR1
(SEQ ID No. 36)
GSSFSINV;
CDR2
(SEQ ID No. 37)
ISWSDGST;
CDR3
(SEQ ID No. 38)
AVEPRGWPKGHRY;
or
(vi)
CDR1
(SEQ ID No. 39)
GSIFRINA;
CDR2
(SEQ ID No. 40)
VNWIGGTT;
CDR3
(SEQ ID No. 41)
SATDKGGSSRY.

The anti-CD123 dAb may comprise one of the sequences shown as SEQ ID No: 42, 43, 44, 45, 46 or 47.

(CD123 dAb H11 45897)
SEQ ID No. 42
QVQLQESGGGLVQAGGSLRLSCTASGRSINTYAMAWFRQAPGKEREFVA
SINYNSRYTHYVDSVKGRFTISRDNTKNTLFLQMDSLNREDTAVYYCAA
TSYYPTDYDVASRVATWPSWGQGTQVTVSS
(CD123 dAb F8 45888)
SEQ ID No. 43
QVQLQESGGGLVQAGESLRLTCAVSGISLNAMGWYRQAPGKQLREWVAV
IKIGGVSNYAVSVKGRFTISRDNAKNTIYLQMNSLKPEDTGVYYCNTYP
PYLNGMDYWGKGTLVTVSS
(CD123 dAb A7 45865)
SEQ ID No. 44
QVQLQQSGGGLVQAGGSLRLSCAFSGRSFNTDAVAWFRQAPGKEREFVA
AISWDGTRTYYADSAKGRFTISRDNAKNTVYLQMNSLNSEDTAVYYCAA
EPQKAWPIGTSAAGFRSWGQGTQVTVSS
(CD123 dAb B4 45868)
SEQ ID No. 45
QVQLQESGGGSVQSGGSLRLSCAASGSSISVMGWFRQAPGKEREFVAAI
SWSDGNTNYADSVNGRFSVSRDNTKNTVYLQMNSLKPEDTAIYYCAVEP
RGWPKGHRYWGQGTQVTVSS
(CD123 dAb A10 45866)
SEQ ID No. 46
QVQLQESGGSSVQAGGSLRLSCAASGSSFSINVMGWFRQAPGKEREFVA
AISWSDGSTNYADSVKGRFTISRDNTKNTVYLQMNSLKPEDTAIYYCAV
EPRGWPKGHRYWGQGTQVTVSS
(CD123 dAb C11 45874)
SEQ ID No. 47
QVQLQESGGGLVQAGGSLRLSCAASGSIFRINAMGWFRQAPGKEREFVT
AVNWIGGTTNYADSVKGRFTISRDNAKNTVYLQMNSLKPEDTAIYFCSA
TDKGGSSRYWGQGTQVTVSS

The present invention also provides:
a CAR comprising such a CD123 dAb as antigen binding domain;
a nucleic acid sequence encoding such a dAb or CAR.

### FLT3

FMS-like tyrosine kinase 3 (FLT3), a receptor tyrosine kinase (RTK), is a membrane-bound receptor with an intrinsic tyrosine kinase domain. FLT3 is composed of a immunoglobulin-like extracellular ligand-binding domain, a transmembrane domain, a juxtamembrane dimerization domain, and highly conserved intracellular kinase domain interrupted by a kinase insert. FLT3 belongs to the class III subfamily of RTKs, which include structurally similar members such as c-FMS, c-KIT, and PDGF receptor. FLT3 is primarily expressed on committed myeloid and lymphoid progenitors with variable expression in the more mature monocytic lineage.

FLT3 expression has been described in lymphohematopoietic organs such as the liver, spleen, thymus, and placenta. In the unstimulated state, FLT3 receptor exists in a monomeric, unphosphorylated form with an inactive kinase moiety. Upon interaction of the receptor with FLT ligand (FL), the receptor undergoes a conformational change, resulting in the unfolding of the receptor and the exposure of the dimerization domain, allowing receptor-receptor dimerization to take place. This receptor dimerization is the prelude to the activation of the tyrosine kinase enzyme, leading to phosphorylation of various sites in the intracellular domain. The amino acid sequence of human FLT3 is available from NCBI Reference Sequence: NP_004110.2.

FLT3 is important to the biology of some cases of AML with mutations in FLT3 being among the most commonly found mutations in this disease. These mutations typically result in constitutive activation. Some cases of AML respond to small-molecule inhibition of FLT3.

Several commercially available antibodies against FLT3 are known, such as A2F10 and BV10A4H2 (ThermoFisher).

The present invention provides a domain antibody (dAb) which binds FLT3 and comprises the following complementarity determining regions:

(i)
CDR1
(SEQ ID No. 72)
GIFKTNY;
CDR2
(SEQ ID No. 73)
GTNDGST;
CDR3
(SEQ ID No. 74)
YGLGH;
(ii)
CDR1
(SEQ ID No. 75)
GTISSIRY;
CDR2
(SEQ ID No. 76)
ITSSGNT;
CDR3
(SEQ ID No. 77)
YTMGY;
(iii)
CDR1
(SEQ ID No. 78)
GIFSTNY;
CDR2
(SEQ ID No. 79)
FTNDGGT;
CDR3
(SEQ ID No. 80)
CGLGH;
(iv)
CDR1
(SEQ ID No. 81)
GSISSIRY;
CDR2
(SEQ ID No. 82)
ITSSGST;
CDR3
(SEQ ID No. 83)
YTMGY;
or
(v)
CDR1
(SEQ ID No. 84)
GIFSTNH;
CDR2
(SEQ ID No. 85)
FTNDGST;
CDR3
(SEQ ID No. 86)
YGLGH.

The anti-FLT3 dAb may comprise one of the sequences shown as SEQ ID No. 87, 88, 89, 90 or 91.

(FLT3 dAb B5)
SEQ ID No. 87
QVQLQQSGGGLVQAGGSLRLSCAASGIFKTNYMAWYRQAPGKQRELVAA
FTNDGSTLYGDSVKGRFTISRDDAKYTVSLQMNSLKPEDTAVYYCYGLG
HWGQGTQVIVSSEPKTPKPQPAAADDDDKEQKLISEEDLNGAAHHHHHH
GAA
(FLT3 dAb G3)
SEQ ID No. 88
QVQLQESGGGLVQAGGSLRLSCAASGTISSIRYMNWYRQAPGKQREVVA
YITSSGNTNYADSVKGRFTISRDNAKNTVYLQMDNLKPEDTAAYYCYTM
GYWGQGTQVTVSSEPKIPQPQPAAADDDDKEQKLISEEDLNGAAHHHHH
HGAA
(FLT3 dAb H5)
SEQ ID No. 89
QVQLQESGGGLVQAGGSLRLSCAASGIFSTNYMVWCRQAPGKQRELVAA
FTNDGGTLYADSLKGRFSISQDNAKNTVLLLMNSLKPEDTAVYYCCGLG
HWGRGTKVTVSSEPKIPQPQPAAADDDDKEQKLISEEDLNGAAHHHHHH
GAA
(FLT3 dAb D12)
SEQ ID No. 90
QVQLQESGGGLVQAGGSLRLSCAASGSISSIRYMNWYRQAPGKQRESVA
WITSSGSTNYADSVQGRFTISRDNAKNTVYLQMDNLKPEDTAVYYCYTM
GYWGQGTQVTVSSEPKIPQPQPAAADDDDKEQKLISEEDLNGAAHHHHH
HGAA
(FLT3 dAb F10)
SEQ ID No. 91
QAQVQLQESGGGLVQAGGSLRLSCAASGIFSTNHMAWYRQAPGKQRELV
AAFTNDGSTLYGDSVKGRFVISRDNAKYTVFLQMNSLKPEDTAVYYCYG
LGHWGQGTQVTVSSEPKTPKPQPAAADDDDKEQKLISEEDLNGAAHHHH
HHGAA

The present invention also provides:
a CAR comprising such a FLT3 dAb as antigen binding domain;
a nucleic acid sequence encoding such a dAb or CAR.

### CLL1

Human C-type lectin-like molecule-1 (CLL-1, MICL or CLEC12A), is a type II transmembrane glycoprotein and member of the large family of C-type lectin-like receptors involved in immune regulation. The intracellular domain of CLL-1 contains an ITIM motif as well as a binding site for PI-3 kinase. The pattern of expression of CLL-1 in hematopoietic cells is restricted; it is found in particular in myeloid cells derived from peripheral blood and bone marrow. The amino acid sequence of human CLL1 is available from Uniprot accession No. Q5QGZ9.

Several antibodies have been described against CLL-1, for example in WO2009051974, WO2013169625, WO2016205200 and WO2016040868.

The present invention provides a domain antibody (dAb) which binds CLL1 and comprises the following complementarity determining regions:

(i)
CDR1
(SEQ ID No. 48)
GFTFGNHD;
CDR2
(SEQ ID No. 49)
IDSGGNVI;
CDR3
(SEQ ID No. 50)
ATDLDSGAESLESVY;
(ii)
CDR1
(SEQ ID No. 51)
GFAFGSAD;
CDR2
(SEQ ID No. 52)
IDSGGNTQ;
CDR3
(SEQ ID No. 53)
TDLDPTTDSLENVY;
(iii)
CDR1
(SEQ ID No. 54)
GRTFSAYF;
CDR2
(SEQ ID No. 55)
INWNGD;;
CDR3
(SEQ ID No. 56)
AADTHGAVGLGSERLYDY;
(iv)
CDR1
(SEQ ID No. 57)
GIGVSSTG;
CDR2
(SEQ ID No. 58)
IDRDGTT;
CDR3
(SEQ ID No. 59)
TVVGDYY;
(v)
CDR1
(SEQ ID No. 60)
GFIFGNYD;
CDR2
(SEQ ID No. 61)
ISSGGNDI;
CDR3
(SEQ ID No. 62)
AADLDPGTDSLDNIH;
or
(vi)
CDR1
(SEQ ID No. 63)
GFTLDYYA;
CDR2
(SEQ ID No. 64)
ISSSDGST;
CDR3
(SEQ ID No. 65)
AEAVYYAGVCVAMYDS

The anti-CLL-1 dAb may comprise one of the sequences shown as SEQ ID No. 66, 67, 68, 69, 70 or 71.

(CLL-1 dAb 44548)
SEQ ID No. 66
QVQLQQSGGGLVQPGGSLRLSCVGSGFTFGNHDMSWVRQAPGKEVEFVA
GIDSGGNVIVYEEVVKGRFTISRDNAKNTLYLQMDGLKPEDAGMYFCAT
DLDSGAESLESVYHGQGTQVTVSS
(CLL-1 dAb 44544)
SEQ ID No. 67
QVQLQESGGGLVESGGSLRISCTGFGFAFGSADMSWVRQAPGKEVEFVA
GIDSGGNTQTYEDTVKGRFTISRDNAKNTLYLQMNSLQSEDAGVYFCAT
DLDPTTDSLENVYHGQGTQVIVSS
(CLL-1 dAb 44538)
SEQ ID No. 68
QVQLQESGGGLVQTGDSLRLSCVASGRTFSAYFMGWFRQAPGKEREFVS
AINWNGDSSWYRDSVKGRFTVSRDNAKNTVYLQMNSLEPEDTAVYYCAA
DTHGAVGLGSERLYDYWGQGTQVTVSS
(CLL-1 dAb 44546)
SEQ ID No. 69
QVQLQESGGGVVQAGGSLRLSCAVSGIGVSSTGMGWSRQTPGKQVELVA
LIDRDGTTNYADTVKGRFTISKDNSKNMVYLQMNSLKPEDTALYHCTVV
GDYYWGQGTQVTVSS
(CLL-1 dAb 44545)
SEQ ID No. 70
QVQLQQSGGGLVQPGGSLRLSCVGSGFIFGNYDMSWVRQAPGKEVEFVA
GISSGGNDIVYEDAVKGRFSISRDNARNTVYLDMASVKPEDAGVYYCAA
DLDPGTDSLDNIHHGQGTQVFVSS
(CLL-1 dAb 44536)
SEQ ID No. 71
QVQLQESGGGLVQPGGSLRLSCAASGFTLDYYAIGWFRQAPGKEREGVS
CISSSDGSTAYADSVKGRFTISRDNAKNSVYLQMNSLKPEDTAVYYCAE
AVYYAGVCVAMYDSWGQGTQVTVSS

The present invention also provides:
a CAR comprising such a CD123 dAb as antigen binding domain;
a nucleic acid sequence encoding such a dAb or CAR.

### SPACER

Classical CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

The spacer may cause two CAR-forming polypeptide chains to dimerise. Two of the polypeptide chains may, for example, comprise one or more suitable cysteine residues to form di-sulphide bridge(s). Commonly used spacers include the IgG1 Fc region, the IgG1 hinge or a human CD8 stalk. A hinge spacer may comprise the sequence shown as SEQ ID No. 92

(hinge spacer)
SEQ ID No. 92
EPKSCDKTHTCPPCP

The spacer may be chosen to suit the target antigen, i.e. the location and orientation of the epitope on the target antigen and the distance of the target epitope from the target cell membrane. In an OR gate, different spacers may be used to suit the different relative locations of the target epitopes and also to prevent cross-pairing between the two CARs.

### TRANSMEMBRANE DOMAIN

The transmembrane domain is the portion of a CAR which spans the membrane. The transmembrane domain may be any protein structure which is thermodynamically stable in a membrane. This is typically an alpha helix comprising of several hydrophobic residues. The transmembrane domain of any transmembrane protein can be used to supply the transmembrane portion of the chimeric receptor. The presence and span of a transmembrane domain of a protein can be determined by those skilled in the art using the TMHMM algorithm (http://www.cbs.dtu.dk/services/TMHMM-2.0/). Alternatively, an artificially designed TM domain may be used.

### ENDODOMAIN

The endodomain is the signal-transmission portion of a CAR. It may be part of or associate with the intracellular domain of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signalling may be needed. Co-stimulatory signals promote T-cell proliferation and survival. There are two main types of co-stimulatory signals: those that belong the Ig family (CD28, ICOS) and the TNF family (OX40, 41 BB, CD27, GITR etc). For example, chimeric CD28 and OX40 can be used with CD3-Zeta to transmit a proliferative/survival signal, or all three can be used together.

The endodomain may comprise:
(i) an ITAM-containing endodomain, such as the endodomain from CD3 zeta; and/or
(ii) a co-stimulatory domain, such as the endodomain from CD28 or ICOS; and/or
(iii) a domain which transmits a survival signal, for example a TNF receptor family endodomain such as OX-40, 4-1 BB, CD27 or GITR.

A number of systems have been described in which the antigen recognition portion is on a separate molecule from the signal transmission portion, such as those described in WO015/150771; WO2016/124930 and WO2016/030691. The cell of the present invention may therefore express a CAR system which comprises an antigen-binding component comprising an antigen-binding domain(s) and a transmembrane domain; which is capable of interacting with a separate intracellular signalling component comprising a signalling domain.

The CAR may comprise a signal peptide so that when it is expressed inside a cell, the nascent protein is directed to the endoplasmic reticulum and subsequently to the cell surface, where it is expressed. The signal peptide may be at the amino terminus of the molecule.

### SUICIDE GENE

The cell of the present invention may also express a suicide gene.

A suicide-gene is a genetically encoded mechanism which allows selective destruction of adoptively transferred cells, such as T-cells, in the face of unacceptable toxicity, such as on-target off-tumour toxicity, cytokine release syndrome (CRS) or neurotoxicity.

When the cells of the present invention are used to treat acute myeloid leukemia (AML), the treatment may cause myeloid aplasia in the patient which may be long-lasting or permanent. It is possible to rescue the patient from this state using an allogeneic transplant, such as an allogeneic hematopoietic stem cell transplantation (alloHSCT). The incorporation of a suicide gene in the CAR-expressing cells also enables the CAR-expressing cells to be deleted if a transplant is used, to prevent CAR mediated graft rejection.

The cells of the present invention may comprise one of the suicide-genes previously tested in clinical studies, such as Herpes Simplex Virus thymidine kinase (HSV-TK) or inducible caspase 9 (iCasp9).

WO2013/153391 describes a compact sort-suicide gene comprising a CD20 epitope which enables cells expressing the polypeptide to be selectively killed using Rituximab. The cells of the present invention may express a suicide gene having the sequence shown as SEQ ID No. 93

SEQ ID No. 93
CPYSNPSLCSGGGGSELPTQGTFSNVSTNVSPAKPTTTACPYSNPSLCS
GGGGSPAPRPPTPAPTIASQPLSLRPEACRPAAGGAVHTRGLDFACDIY
IWAPLAGTCGVLLLSLVITLYCNHRNRRRVCKCPRPVV

WO2016/135470 describes a suicide gene which dimerizes in the presence of a chemical inducer of dimerization (CID) such as rapamycin or a rapamycin analogue causing caspase-mediated apoptosis of the cell.

The suicide gene may have the structure:

Ht1-HT2-Casp
in which:
Ht1 and Ht2 are heterodimerisation domains, one of which comprises an FK506-binding protein (FKBP) and the other of which comprises an FRB domain of mTOR; and
Casp is a caspase 9 domain.

The suicide gene may have the sequence shown as SEQ ID No. 94 or a variant thereof having 90, 95, or 99% sequence identity.

(FRB-FKBP12-L3-dCasp9)
SEQ ID No. 94
<-----------------------FRB---------------------------------
MASRILWHEMWHEGLEEASRLYFGERNVKGMFEVLEPLHAMMERGPQTLKETSFNQAYGR
-----------------FRB-------------------><L1-><--FKBP12------
DLMEAQEWCRKYMKSGNVKDLLQAWDLYYHVFRRISKLEYSGGGSLEGVQVETISPGDGR
------------------FKBP12------------------------------------
TFPKRGQTCVVHYTGMLEDGKKFDSSRDRNKPFKFMLGKQEVIRGWEEGVAQMSVGQRAK
---------------------------------><------L3------><--dCasp9-
LTISPDYAYGATGHPGIIPPHATLVFDVELLKLESGGGGSGGGGSGGGGSGVDGFGDVGA
------------------------dCasp9------------------------------
LESLRGNADLAYILSMEPCGHCLIINNVNFCRESGLRTRTGSNIDCEKLRRRFSSLHFMV
------------------------dCasp9------------------------------
EVKGDLTAKKMVLALLELAQQDHGALDCCVVVILSHGCQASHLQFPGAVYGTDGCPVSVE
------------------------dCasp9------------------------------
KIVNIFNGTSCPSLGGKPKLFFIQACGGEQKDHGFEVASTSPEDESPGSNPEPDATPFQE
------------------------dCasp9------------------------------
GLRTFDQLDAISSLPTPSDIFVSYSTFPGFVSWRDPKSGSWYVETLDDIFEQWAHSEDLQ
--------------dCasp9---------------->
SLLLRVANAVSVKGIYKQMPGCFNFLRKKLFFKTSAS

### NUCLEIC ACID CONSTRUCT

A nucleic acid sequence encoding a CAR may have the following structure:

Ag B-spacer-TM-endo
in which:

AgB is a nucleic acid sequence encoding an antigen binding domain of the CAR;
spacer is a nucleic acid sequence encoding a spacer of the CAR;
TM is a nucleic acid sequence encoding a transmembrane domain of the CAR;
endo is a nucleic acid sequence encoding an endodomain of the CAR.

A nucleic acid sequence encoding a tanCAR may have the following structure:

AgB1-linker-AgB2-spacer-TM-endo
in which
AgB1 is a nucleic acid sequence encoding a first antigen-binding domain of the tanCAR;
linker is a nucleic acid sequence encoding a linker of the tanCAR;
AgB2 is a nucleic acid sequence encoding the second antigen binding domain of the tanCAR;
spacer is a nucleic acid sequence encoding a spacer of the tanCAR;
TM is a nucleic acid sequence encoding a transmembrane domain of the tanCAR;
endo is a nucleic acid sequence encoding an endodomain of the tanCAR.

When expressed in a cell, the nucleotide sequence encodes a polypeptide expressing the first and second antigen binding domains in tandem at the cell surface.

The linker may be or comprise a Gly-Ser flexible linker.

The antigen binding domain(s) for the CAR or tanCAR may, for example, be scFv(s) or dAb(s).

The present invention provides a nucleic acid construct encoding a triple OR gate, which comprises three CARs.

A nucleic acid construct encoding a triple OR gate may have the structure:

AgBD1-spacer1 -TM1-endo1-coexpr1 -AgBD2-spacer2-TM2-endo2-coexpr2-AgBD3-spacer3-TM3-endo3
in which:
AgBD1 is a nucleic acid sequence encoding an antigen binding domain of the first CAR;
Spacer1 is a nucleic acid sequence encoding a spacer of the first CAR;
TM1 is a a nucleic acid sequence encoding a transmembrane domain of the first CAR;
Endo1 is a nucleic acid sequence encoding an endodomain of the first CAR;; coexpr1 and coexpr2, which may be the same or different, are nucleic acid sequences enabling co-expression the first, second and third CARs;
AgBD2 is a nucleic acid sequence encoding an antigen binding domain of the second CAR;
Spacer2 is a nucleic acid sequence encoding a spacer of the second CAR;
TM2 is a a nucleic acid sequence encoding a transmembrane domain of the second CAR;
Endo2 is a nucleic acid sequence encoding an endodomain of the second CAR;
AgBD3 is a nucleic acid sequence encoding an antigen binding domain of the third CAR;
Spacer3 is a nucleic acid sequence encoding a spacer of the third CAR;
TM3 is a a nucleic acid sequence encoding a transmembrane domain of the third CAR; and
Endo3 is a nucleic acid sequence encoding an endodomain of the third CAR.

The antigen-binding domain of the first, second and third CARs may, for example, be an scFv or a dAb. In particular, all three CARs may have a dAb antigen binding domain.

The present invention provides a nucleic acid construct encoding a quadruple OR gate, which comprises four CARs.

A nucleic acid construct encoding a quadruple OR gate may have the structure:

AgBD1-spacer1 -TM1-endo1-coexpr1 -AgBD2-spacer2-TM2-endo2-coexpr2-Ag BD3-spacer3-TM3-endo3- coexpr3-AgBD4-spacer4-TM4-endo4
in which:
AgBD1 is a nucleic acid sequence encoding an antigen binding domain of the first CAR;
Spacer1 is a nucleic acid sequence encoding a spacer of the first CAR;
TM1 is a a nucleic acid sequence encoding a transmembrane domain of the first CAR;
Endo1 is a nucleic acid sequence encoding an endodomain of the first CAR; coexpr1, coexpr2 and coexpr3, which may be the same or different, are nucleic acid sequences enabling co-expression the first, second, third and fourth CARs;
AgBD2 is a nucleic acid sequence encoding an antigen binding domain of the second CAR;
spacer2 is a nucleic acid sequence encoding a spacer of the second CAR;
TM2 is a a nucleic acid sequence encoding a transmembrane domain of the second CAR;
endo2 is a nucleic acid sequence encoding an endodomain of the second CAR;
AgBD3 is a nucleic acid sequence encoding an antigen binding domain of the third CAR;
spacer3 is a nucleic acid sequence encoding a spacer of the third CAR;
TM3 is a a nucleic acid sequence encoding a transmembrane domain of the third CAR; and
endo3 is a nucleic acid sequence encoding an endodomain of the third CAR;
AgBD4 is a nucleic acid sequence encoding an antigen binding domain of the fourth CAR;
spacer4 is a nucleic acid sequence encoding a spacer of the fourth CAR;
TM4 is a a nucleic acid sequence encoding a transmembrane domain of the fourth CAR; and
endo4 is a nucleic acid sequence encoding an endodomain of the fourth CAR.

The antigen-binding domain of the first, second, third and fourth CARs may, for example, be an scFv or a dAb. In particular, all four CARs may have a dAb antigen binding domain.

The present invention also provides a nucleic acid construct encoding an scFv/dAb CAR and a tanCAR. In this embodiment, the nucleic acid construct may have the structure:

AgB1 -linker-Ag B2-spacer1-TM1-endo1-coexpr-AgB3-spacer2-TM2-endo2
in which:
AgB1 is a nucleic acid sequence encoding the first antigen-binding domain of the tanCAR;
linker is a nucleic acid sequence encoding a linker of the tanCAR;
AgB2 is a nucleic acid sequence encoding the second antigen binding domain of the tanCAR;
spacer1 is a nucleic acid sequence encoding a spacer of the tanCAR;
TM1 is a nucleic acid sequence encoding a transmembrane domain of the tanCAR;
endo1 is a nucleic acid sequence encoding an endodomain of the tanCAR;
coexpr is a nucleic acid sequence enabling co-expression the CAR and the tanCAR;
AgB3 is a nucleic acid sequence encoding the antigen-binding domain of the CAR
spacer2 is a nucleic acid sequence encoding a spacer of the CAR;
TM2 is a nucleic acid sequence encoding a transmembrane domain of the CAR;
endo2 is a nucleic acid sequence encoding an endodomain of the CAR;
or the structure:
AgB1-spacer1-TM1-endol -coexpr-AgB2-linker-AgB3-spacer2-TM2-endo2
in which:
AgB1 is a nucleic acid sequence encoding the antigen-binding domain of the CAR spacer1 is a nucleic acid sequence encoding a spacer of the CAR;
TM1 is a nucleic acid sequence encoding a transmembrane domain of the CAR;
endo1 is a nucleic acid sequence encoding an endodomain of the CAR;
coexpr is a nucleic acid sequence enabling co-expression the CAR and the tanCAR;
AgB2 is a nucleic acid sequence encoding the first antigen-binding domain of the tanCAR;
linker is a nucleic acid sequence encoding a linker of the tanCAR;
AgB3 is a nucleic acid sequence encoding the second antigen binding domain of the tanCAR;
spacer2 is a nucleic acid sequence encoding a spacer of the tanCAR;
TM2 is a nucleic acid sequence encoding a transmembrane domain of the tanCAR;
endo2 is a nucleic acid sequence encoding an endodomain of the tanCAR.

The present invention also provides a nucleic acid construct encoding two tanCARs. In this embodiment, the nucleic acid construct may have the structure:

AgB1 -linker1 -Ag B2-spacer1-TM1 -endo1 -coexpr-Ag B3-linker2-Ag B4-spacer2-TM2-endo2
in which:
AgB1 is a nucleic acid sequence encoding the first antigen-binding domain of the first tanCAR;
linker1 is a nucleic acid sequence encoding a linker of the first tanCAR;
AgB2 is a nucleic acid sequence encoding the second antigen binding domain of the first tanCAR;
spacer1 is a nucleic acid sequence encoding a spacer of the first tanCAR;

TM1 is a nucleic acid sequence encoding a transmembrane domain of the first tanCAR;
endo1 is a nucleic acid sequence encoding an endodomain of the first tanCAR;

coexpr is a nucleic acid sequence enabling co-expression the first and second tanCARs;
AgB3 is a nucleic acid sequence encoding the first antigen-binding domain of the second tanCAR;
linker2 is a nucleic acid sequence encoding a linker of the second tanCAR;
AgB4 is a nucleic acid sequence encoding the second antigen binding domain of the second tanCAR;
spacer2 is a nucleic acid sequence encoding a spacer of the second tanCAR;
TM2 is a nucleic acid sequence encoding a transmembrane domain of the second tanCAR;
endo2 is a nucleic acid sequence encoding an endodomain of the second tanCAR.

The nucleic acid construct of the present invention may also comprise a nucleic acid sequence encoding a suicide gene.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

In the structure above, “coexpr” is a nucleic acid sequence enabling co-expression of two polypeptides as separate entities. It may be a sequence encoding a cleavage site, such that the nucleic acid construct produces both polypeptides, joined by a cleavage site(s). The cleavage site may be self-cleaving, such that when the polypeptide is produced, it is immediately cleaved into individual peptides without the need for any external cleavage activity.

The cleavage site may be any sequence which enables the two polypeptides to become separated.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may, for example be a furin cleavage site, a Tobacco Etch Virus (TEV) cleavage site or encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al (2001) as above).

The cleavage site may comprise the 2A-like sequence shown as SEQ ID No. 95

SEQ ID No. 95:
RAEGRGSLLTCGDVEENPGP

### VECTOR

The present invention also provides a vector, or kit of vectors, which comprises one or more nucleic acid sequence(s) encoding one or more chimeric antigen receptor(s) of the cell of the invention. Such a vector may be used to introduce the nucleic acid sequence(s) into a host cell so that it expresses the or each CAR(s).

The vector may, for example, be a plasmid or a viral vector, such as a retroviral vector or a lentiviral vector, or a transposon based vector or synthetic mRNA.

The vector may be capable of transfecting or transducing a cell such as a T cell or a NK cell.

### CELL

The present invention provides a cell which comprises a plurality of chimeric antigen receptors.

The cell may be a cytolytic immune cell such as a T cell or an NK cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell- mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The cell may be a Natural Killer cell (or NK cell). NK cells form part of the innate immune system. NK cells provide rapid responses to innate signals from virally infected cells in an MHC independent manner NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The cells of the invention may be any of the cell types mentioned above.

T or NK cells according to the first aspect of the invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, T or NK cells according to the first aspect of the invention may be derived from ex vivo differentiation of inducible progenitor cells or embryonic progenitor cells to T or NK cells. Alternatively, an immortalized T-cell line which retains its lytic function and could act as a therapeutic may be used.

In all these embodiments, chimeric polypeptide-expressing cells are generated by introducing DNA or RNA coding for the chimeric polypeptide by one of many means including transduction with a viral vector, transfection with DNA or RNA.

The cell of the invention may be an ex vivo T or NK cell from a subject. The T or NK cell may be from a peripheral blood mononuclear cell (PBMC) sample. T or NK cells may be activated and/or expanded prior to being transduced with nucleic acid encoding the molecules providing the chimeric polypeptide according to the first aspect of the invention, for example by treatment with an anti-CD3 monoclonal antibody.

The T or NK cell of the invention may be made by:
(i) isolation of a T or NK cell-containing sample from a subject or other sources listed above; and
(ii) transduction or transfection of the T or NK cells with a nucleic acid construct, vector or kit of vectors of the invention.

The T or NK cells may then by purified, for example, selected on the basis of expression of the antigen-binding domain of the antigen-binding polypeptide.

### PHARMACEUTICAL COMPOSITION

The present invention also relates to a pharmaceutical composition containing a plurality of cells according to the invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### METHOD OF TREATMENT

The present invention provides a method for treating a disease which comprises the step of administering the cells of the present invention (for example in a pharmaceutical composition as described above) to a subject.

A method for treating a disease relates to the therapeutic use of the cells of the present invention. Herein the cells may be administered to a subject having an existing disease or condition in order to lessen, reduce or improve at least one symptom associated with the disease and/or to slow down, reduce or block the progression of the disease.

The method may involve the steps of:
(i) isolating a T or NK cell-containing sample;
(ii) transducing or transfecting such cells with a nucleic acid sequence or vector provided by the present invention;
(iii) administering the cells from (ii) to a subject.

The T or NK cell-containing sample may be isolated from a subject or from other sources, for example as described above. The T or NK cells may be isolated from a subject's own peripheral blood (1st party), or in the setting of a haematopoietic stem cell transplant from donor peripheral blood (2nd party), or peripheral blood from an unconnected donor (3rd party).

The method may involve the following steps:
(i) administering cells of the first aspect of the invention to a subject;
(ii) administering an allogeneic hematopoietic stem cell transplant (alloHSCT) to the subject.

For example, the method may involve the following steps:
(i) administering cells of the first aspect of the invention to a subject;
(ii) monitoring the subject for myeloid aplasia
(ii) administering an allogeneic hematopoietic stem cell transplant (alloHSCT) to the patient if myeloid aplasia is detected.

Myeloid metaplasia is a clinical and pathologic syndrome which is characterized by the constant occurrence of extramedullary hematopoiesis in the spleen and almost always in the liver, splenomegaly and usually hepatomegaly, and an anemia with immature red and white cells in the peripheral blood.

The present invention provides a cell of the present invention for use in treating and/or preventing a disease.

The invention also relates to the use of a cell of the present invention in the manufacture of a medicament for the treatment and/or prevention of a disease.

The disease to be treated by the m Acute Myeloid Leukemia (AML) ethods of the present invention may be a cancerous disease. In particular, the disease may be Acute Myeloid Leukemia (AML).

Acute myeloid leukemia (AML) is a cancer of the myeloid line of blood cells, characterized by the rapid growth of abnormal cells that build up in the bone marrow and blood and interfere with normal blood cells. Symptoms may include feeling tired, shortness of breath, easy bruising and bleeding, and increased risk of infection. Diagnosis is usually based on bone marrow aspiration and blood tests As an acute leukemia, AML progresses rapidly and is typically fatal within weeks or months if left untreated.

The cells of the present invention may be capable of killing target cells, such as cancer cells. The target cell may be characterised by the expression of one or more target antigens, such as one, two, three or all four of the following: CD33, CD123, CLL-1 and FLT3.

The cells and pharmaceutical compositions of present invention may be for use in the treatment and/or prevention of the diseases described above.

The invention will now be further described by way of Examples, which are meant to serve to assist one of ordinary skill in the art in carrying out the invention and are not intended in any way to limit the scope of the invention.

### EXAMPLES

### Example 1—Designing and Generation Cells Expressing Single CARs and Triple OR Gates

A panel of CAR-encoding nucleic acid constructs are generated as follows:
RQR8-2A-V5CD123CAR—single CAR construct expressing CD123 CAR with V5 tag
RQR8-2A-V5FLT3CAR—single CAR construct expressing FLT3 CAR with V5 tag
RQR8-2A-HACD33CAR—single CAR construct expressing CD33 CAR with HA tag
RQR8-2A-FLAGCLL1CAR—single CAR construct expressing CLL1 CAR with FLAG tag

RQR8-2A-V5CD123CAR-2A-HACD33CAR-2A-FLAGCLL1CAR-
triple CAR construct expressing CD123 CAR with V5
V5 tag; CD33 CAR with HA tag; and CLL1 CAR with
FLAG tag
RQR8-2A-V5FLT3CAR-2A-HACD33CAR-2A-FLAGCLL1CAR-
triple CAR construct expressing FLT3 CAR with V5
tag; CD33 CAR with HA tag; and CLL1 CAR with
FLAG tag

All constructs co-express the sort-suicide gene RQR8, which is described in WO2013/153391.

All CARs are dAb CARs having a second generation endodomain comprising CD3 and a 4-1 BB co-stimulatory domain.

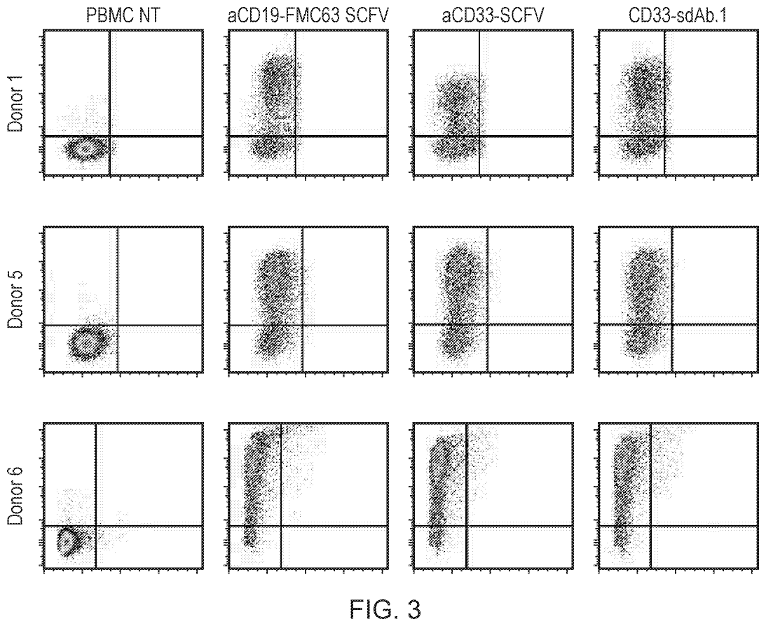

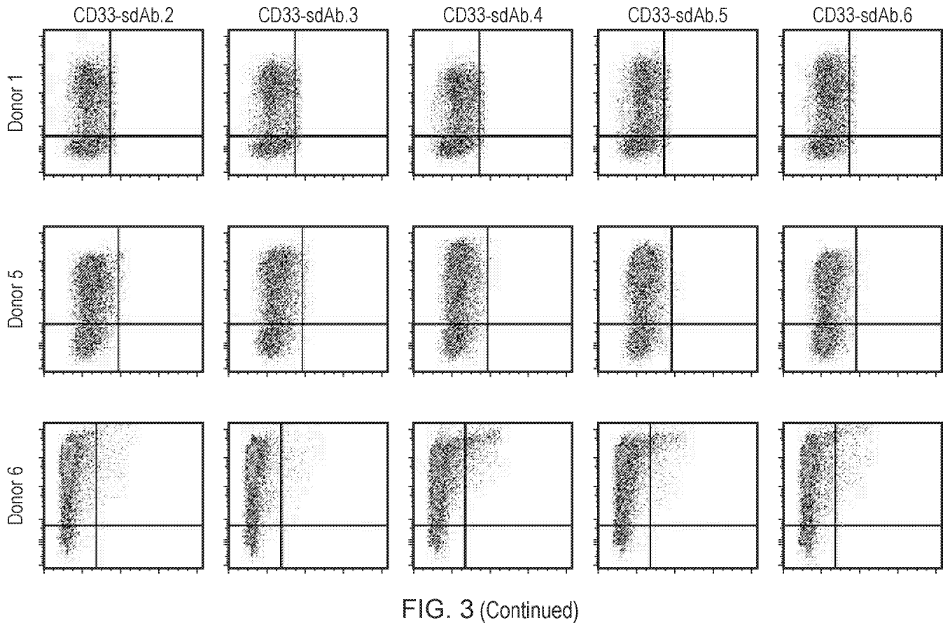

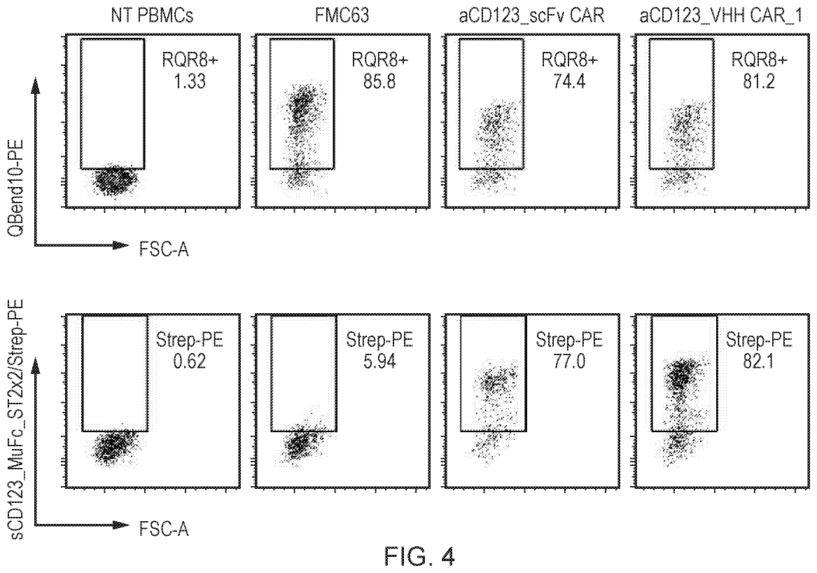

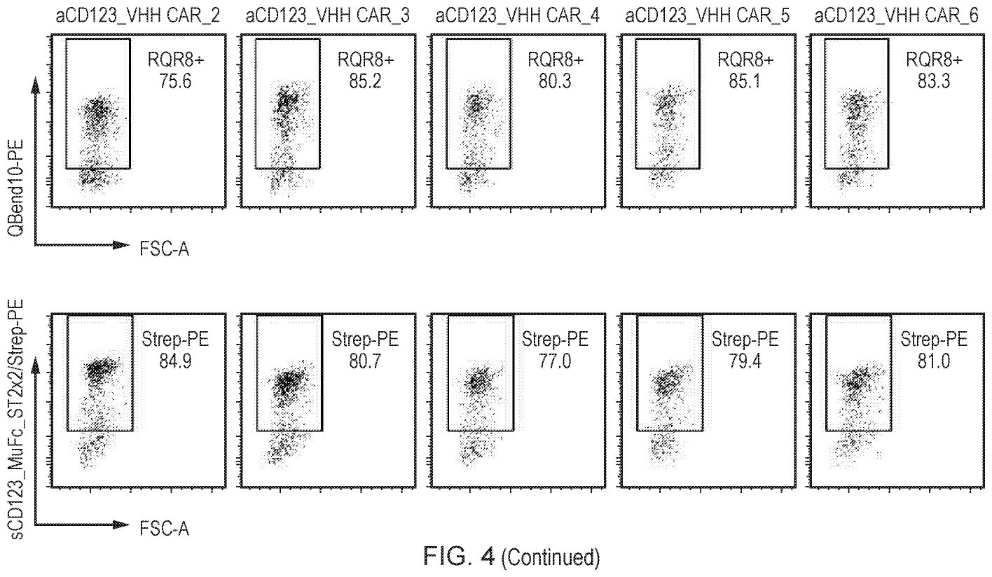

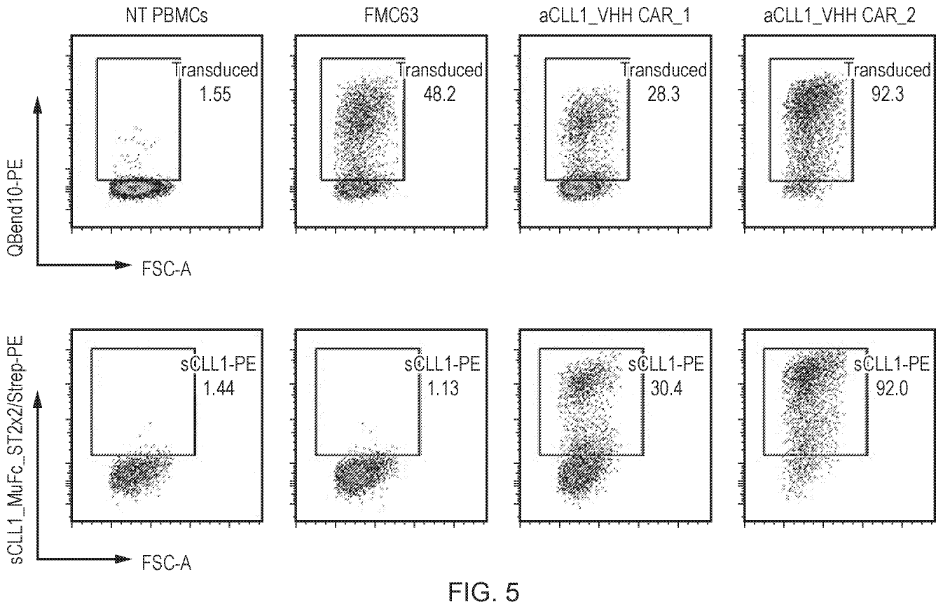

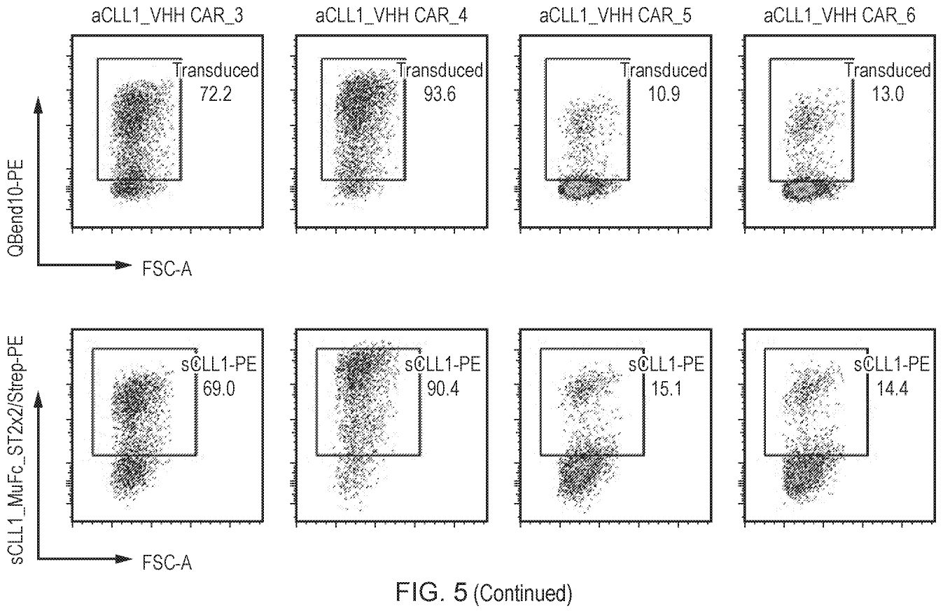

Peripheral blood-derived CD4+ and CD8+ T cells were transduced with the constructs. Expression of the single CARs detected by co-staining T cells with the CAR-specific tag antibody (against V5, HA or FLAG) and QBEND10 (to detect expression of RQR8). FIGS. 3 shows expression of aCD33 CAR. FIG. 4 shows expression of aCD123 CAR. FIG. 5 shows expression of aCLL1 CAR.

Expression of the triple CARs is detected by co-staining T cells with all three CAR-specific tag antibodies (against V5, HA and FLAG) and QBEND10 (to detect expression of RQR8).

### Example 2—FACs-Based Killing Assay (FBK)

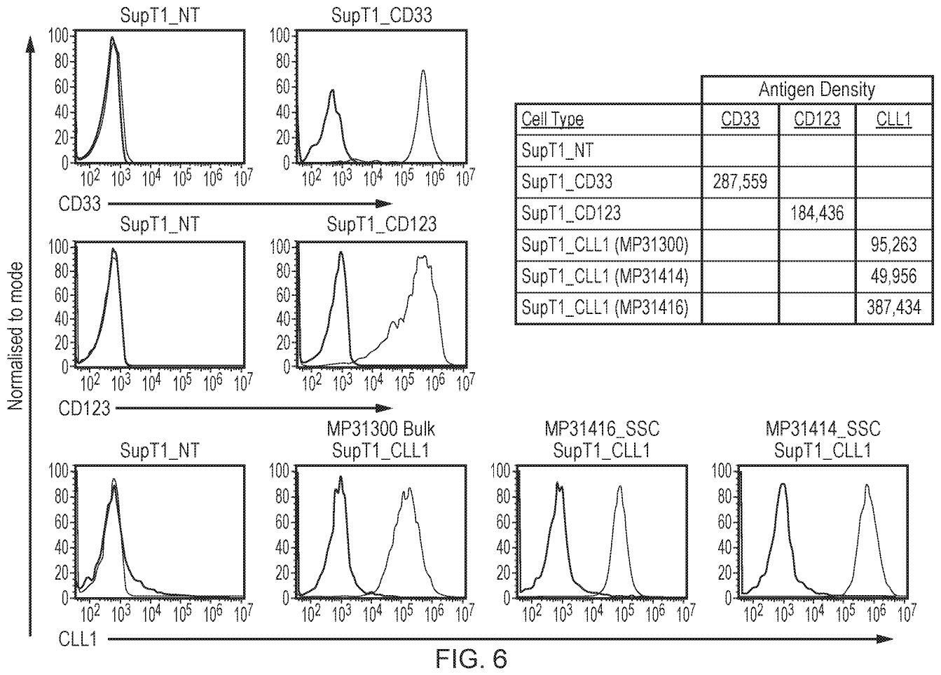

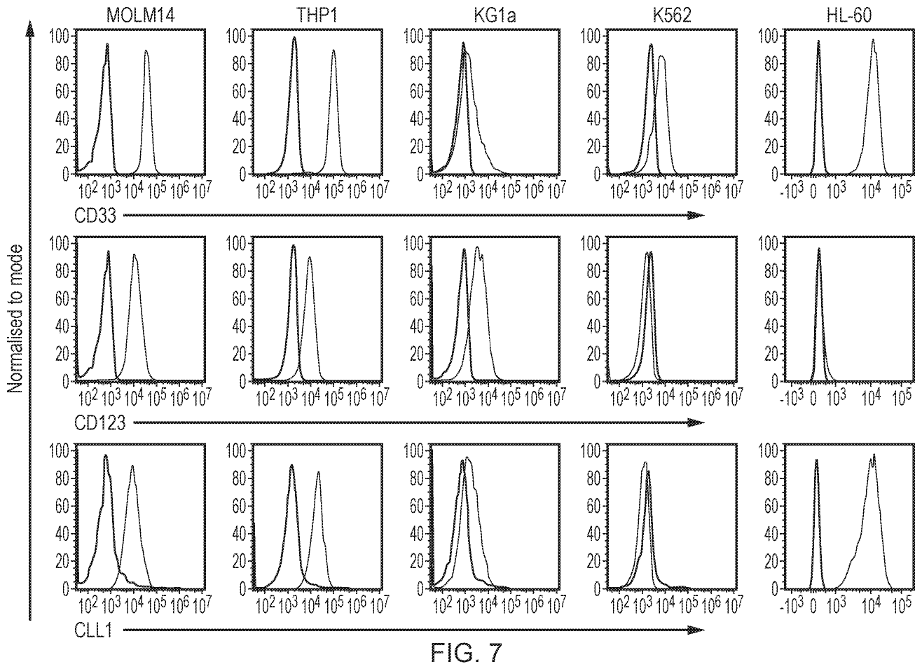

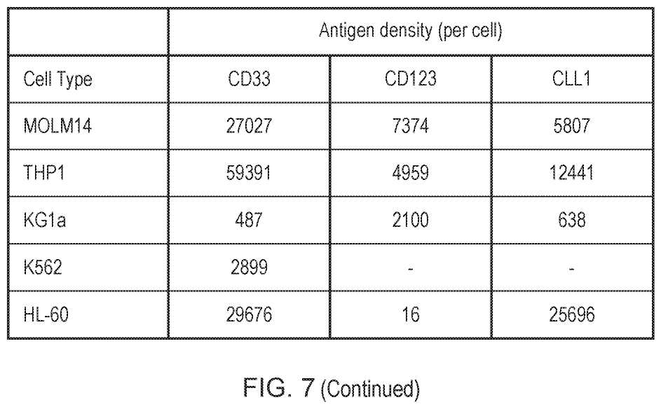

The capacity of the cells expressing single CARs to kill target cells expressing the individual antigens CD123, CLL1, and CD33 was investigated using a FACS-based killing assay. The assay was conducted using SupT1 cell lines engineered to express the desired antigen and with human, patient-derived cell lines from literature (Molm- 14, KG1α, HL-60, K562, and THP-1), which were investigated for uniform antigen expression prior to use (FIGS. 6 and 7).

T cells were co-cultured with the target cells at a ratio of 1:1. The assay was carried out in a 96-well plate in 0.2 ml total volume using 5×10⁴ transduced T-cells per well and target cells in a ratio of 1:1, 1:2, 1:4, or 1:8. The co-cultures were set up after being normalised for the transduction efficiency. The FBK was carried out after 24 or 48 h of incubation.

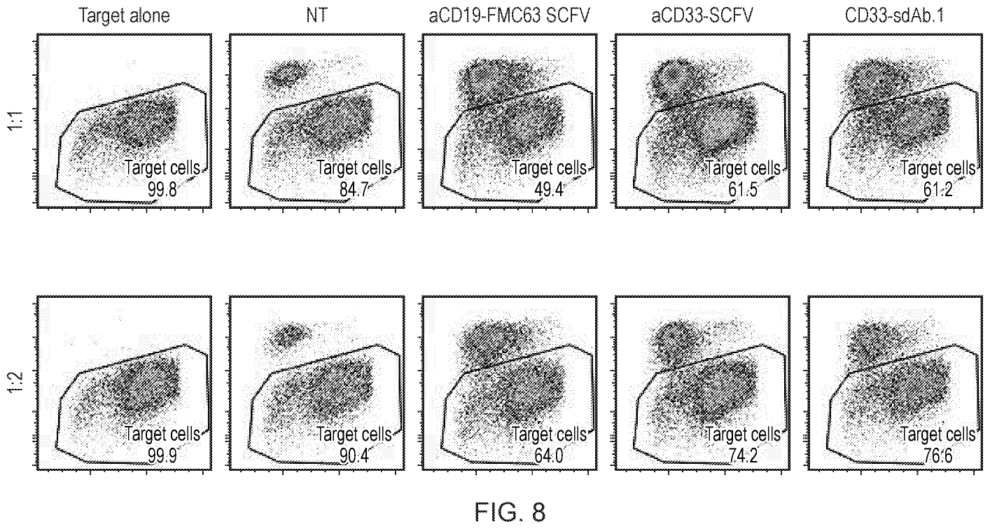

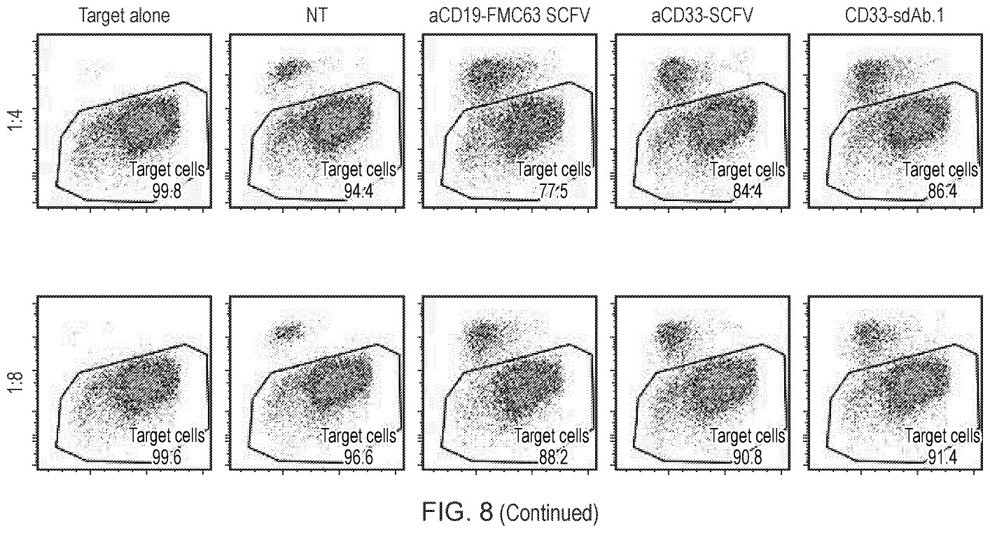

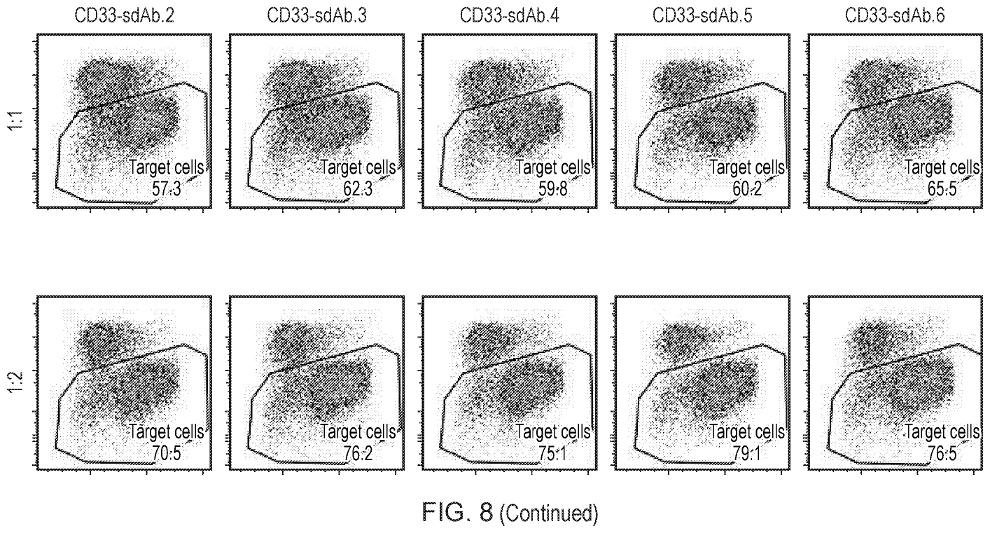

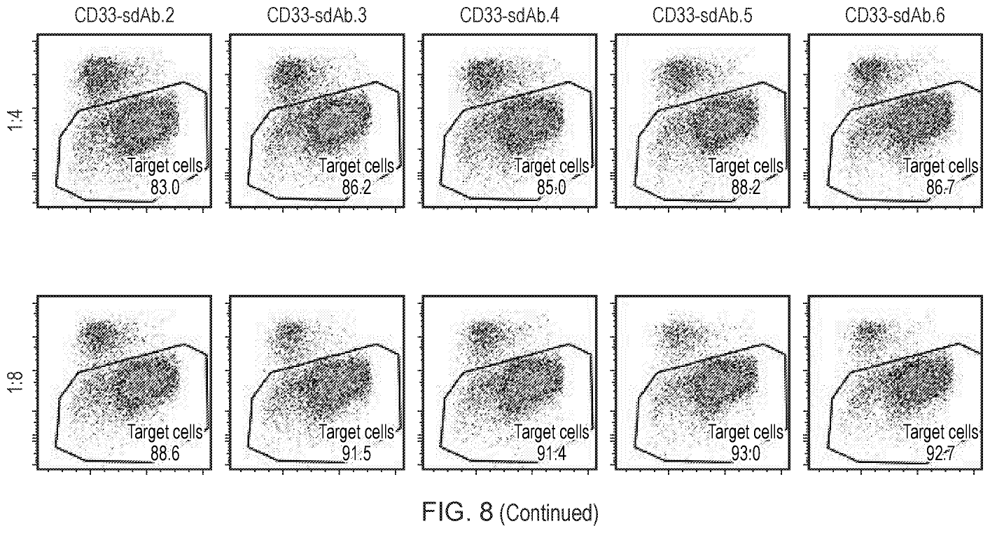

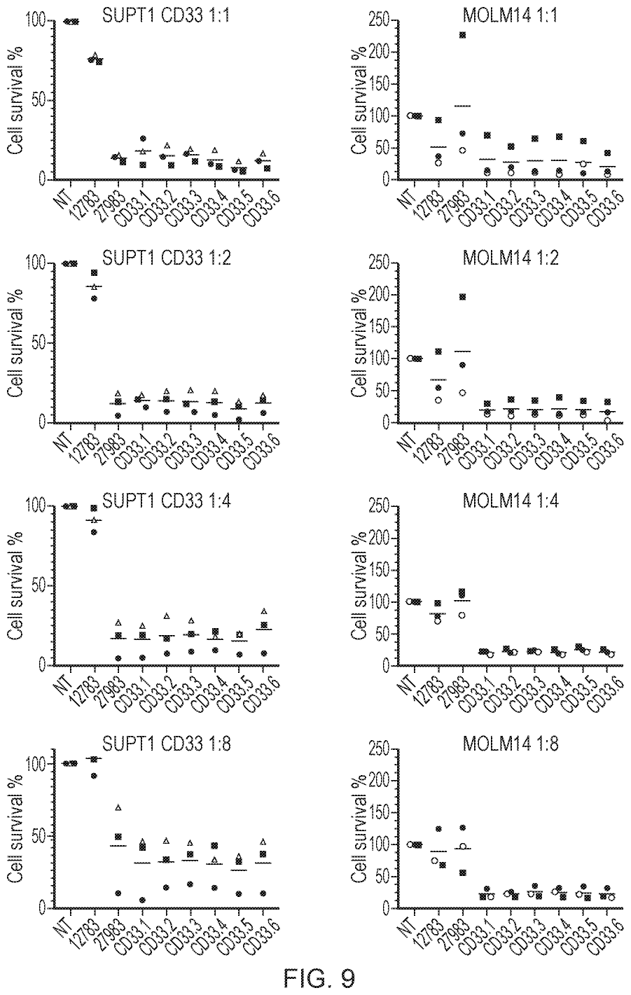

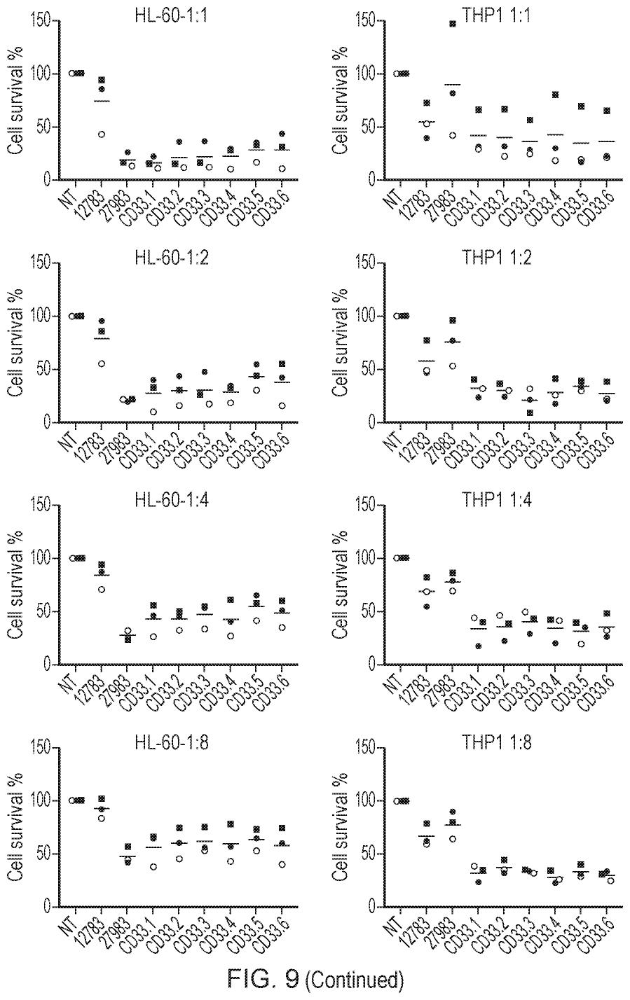

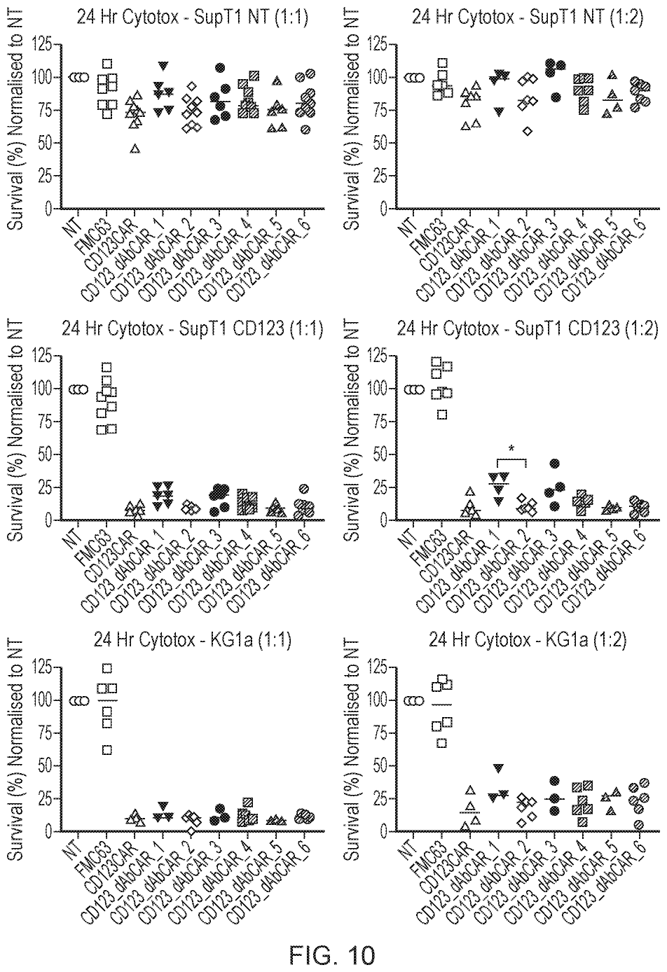

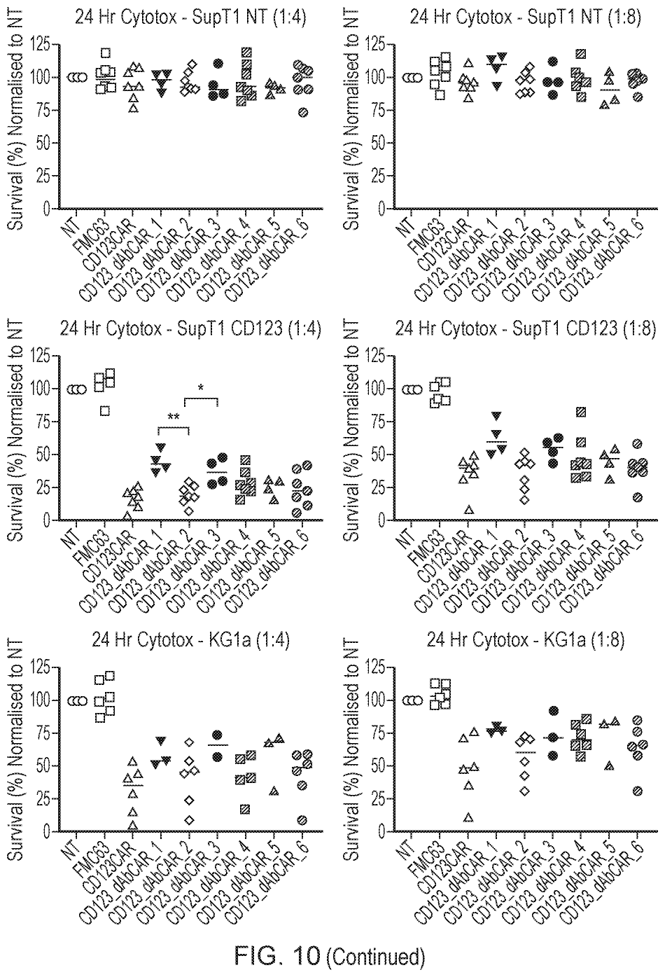

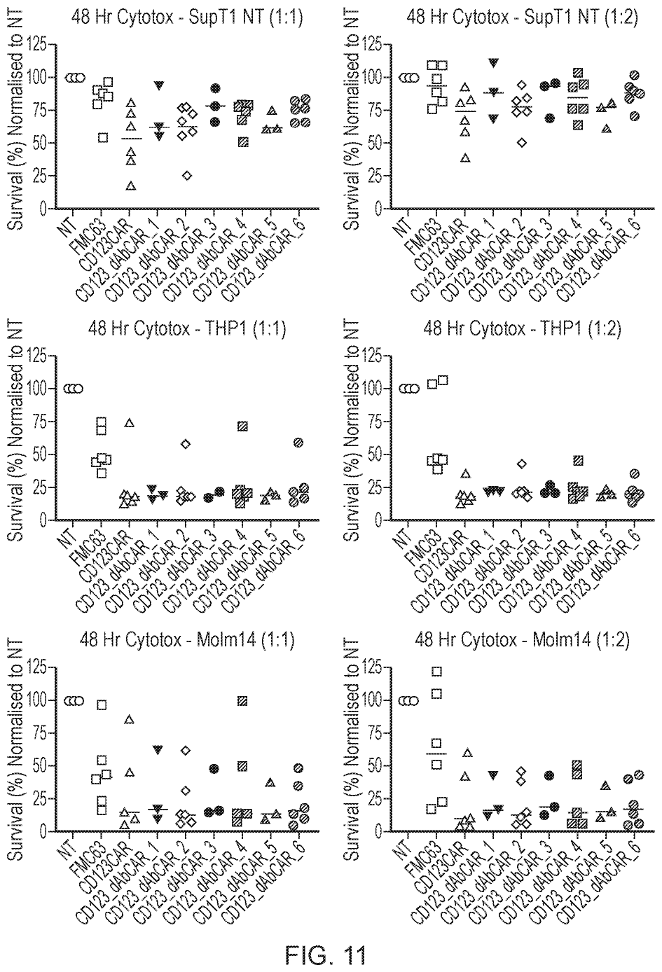

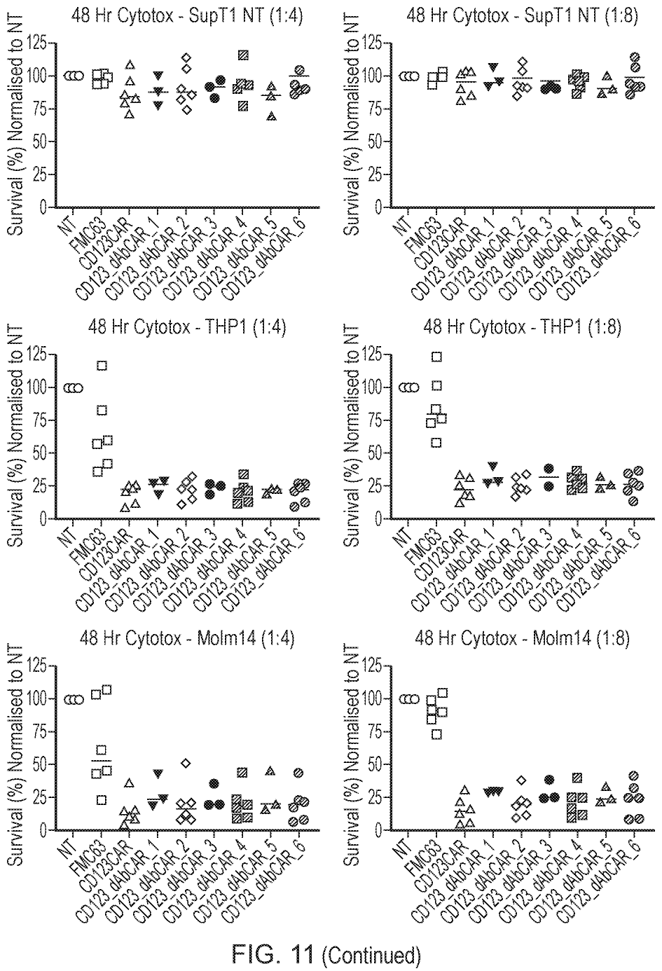

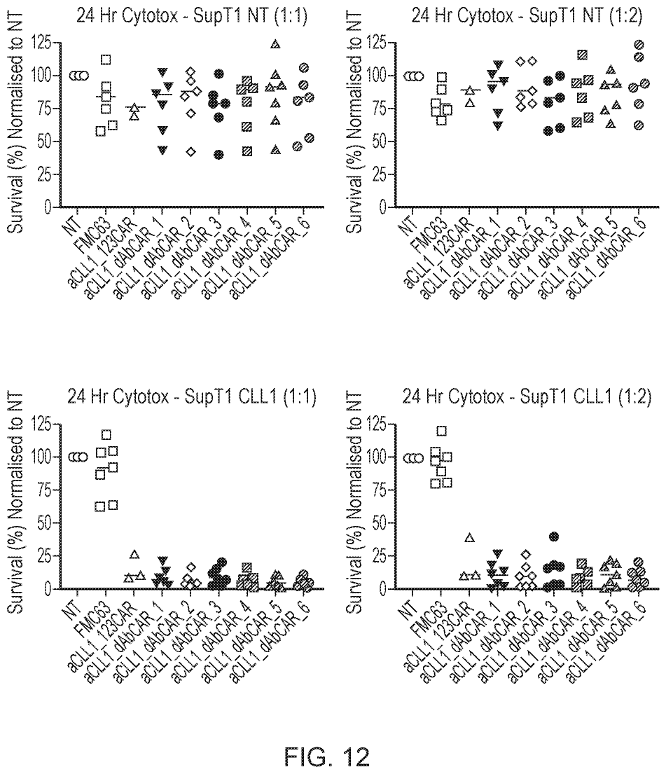

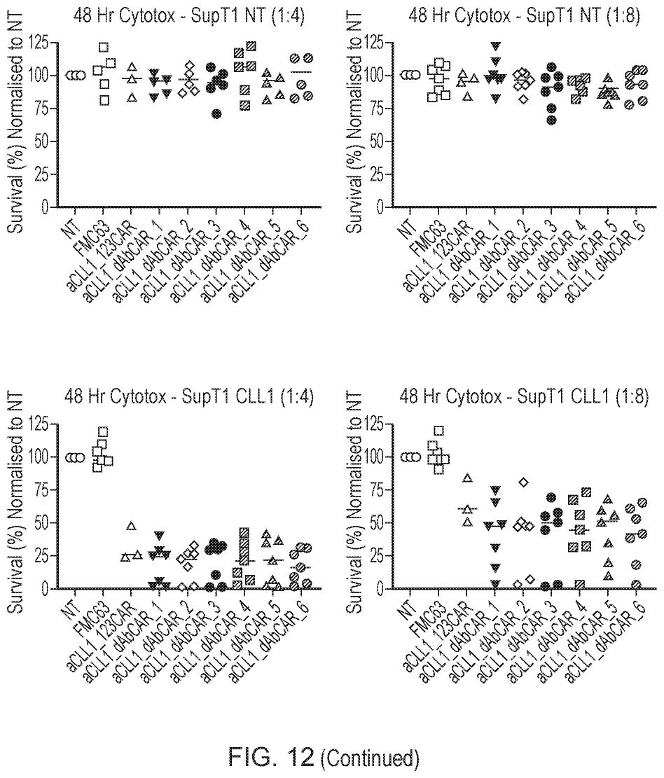

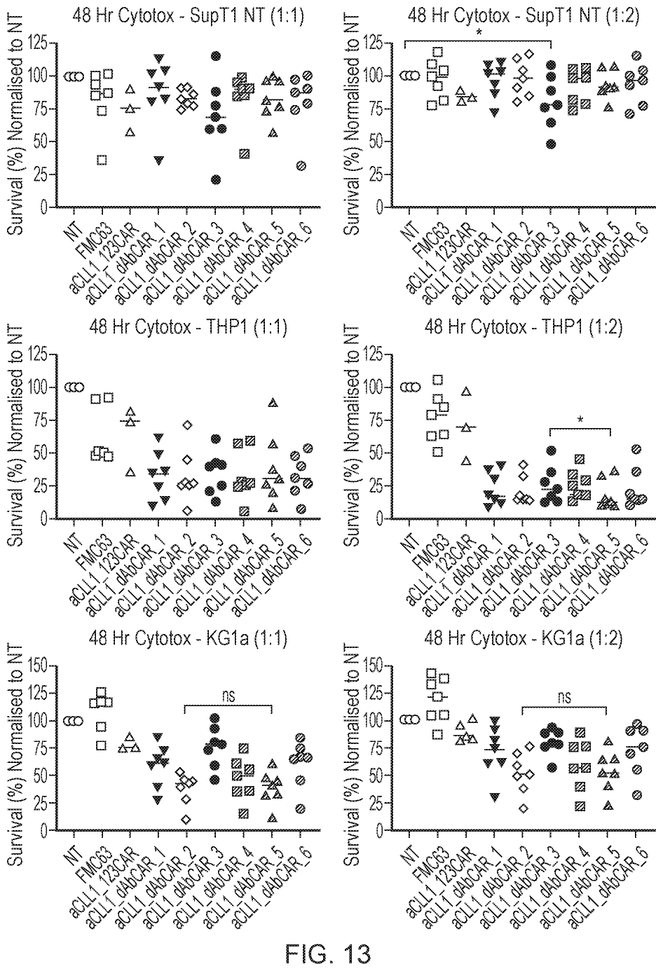

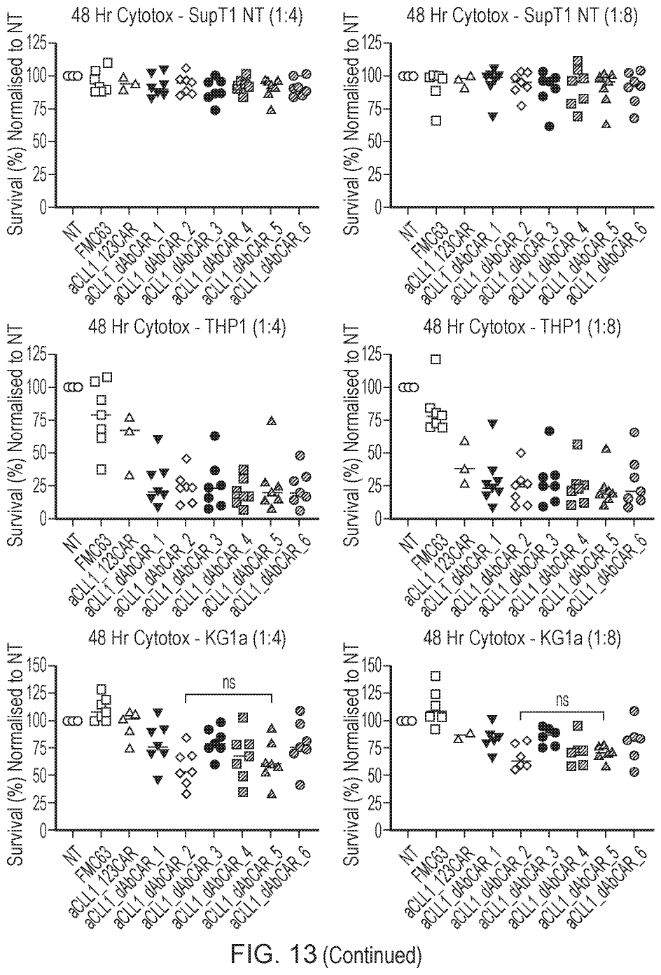

The results of the FBK are shown in FIGS. 8 and 9 for aCD33, FIGS. 10 and 11 for aCD123, and FIGS. 12 and 13 for aCLL1.

All single aCD33 dab CARs showed potent cytotoxic activity on each AML cells expressing CD33 compared to the negative control. The dose response killing was as expected with the higher effector and target ratio (1:1) leading to a higher target killing. aCD19 FMC63 CAR showed some level of low background cytotoxic activity in some cells. No major significant difference in the cytotoxic level were observed between the CD33 single domain CARs. A killing response around 50-60% was seen even with the lowest E:T ratio in all cells.

CD123-VHH-CAR-2 displayed significant difference over CD123-VHH-CAR_1 against SupT1 CD123 at 1:2 and 1:4 (p=0.0205 N=4 and p=0.0012 n=4, respectively). Whilst a significant difference could only be seen for CD123-VHH-CAR_2 over CD123-VHH-CAR_3 against SupT1 CD123 for 1:4 (p=0.0194, n=4). For the remaining 24 hour co-cultures, there was potent CD123 specific cytotoxic activity across all CD123 CARs with no significant difference between constructs across all ratios (FIG. 10). However, there was significant antigen-independent basal cytotoxicity observed for CD123-/SupT1 NT cells at 1:1 ratio only. As expected, a dose-response behaviour was displayed for greater target survival with increasing E:T ratios. All constructs displayed 70% target lysis on SupT1 CD123 and KG1a cell lines in the absence of basal antigen-independent cytotoxicity (E:T, 1:2). For the 48 hour co-cultures, there was significant non-specific cytotoxicity for SupT1 NT with ratios 1:1 and 1:2 across all constructs. At 1:8 there was no significant basal cytotoxicity against CD123-/SupT1 NT for all CAR constructs, with all CAR constructs achieving 70% target lysis for Molm14 and THP1 (FIG. 11).

No significant difference was observed for SupT1 CLL1 with all VHH binders displaying potent CLL1 specific cytotoxicity (FIG. 12). This trend was exhibited across all E:T ratios with no single VHH displaying enhanced activity. Basal activity was significant for all CARs incubated with CLL1-/SupT1 cells for 48 hours at a 1:1 ratio, with only CLL1-VHH-CAR-3 displaying significant basal activity for the 1:2 E:T ratio (p=0.0425, n=7). All CARs displayed CLL specific cytotoxicity when incubated with THP1 and KG1a cells, with VHH-CARs 2 and 5 displaying enhanced killing VHH CARs (FIG. 13). The only CAR to display significant CLL1 specific cytotoxicity for THP1 was CAR-5 over CAR −3 (p=0.0133, n=7) at a 1:2 E:T ratio. There was no significant difference in CLL1 specific cytotoxicity between CARs 2 and 5 across all ratios with all cell lines.

### Example 3—Cytokine Release

IL-2 secretion is an indicator of T cell activation and was evaluated by using the supernatant of the cytotoxic co-culture assays. Cytokine secretion was analysed for single CARs alongside relevant controls, investigating IL-2 and IFNγ production as markers of T-cell activation. Production of IL-2 and IFNγ was detected by ELISA.

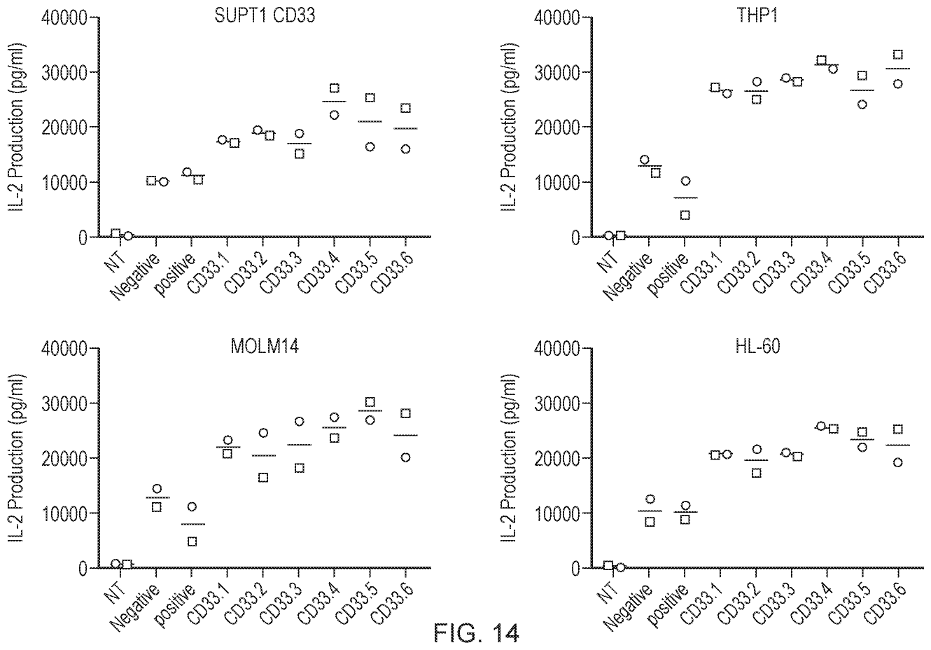

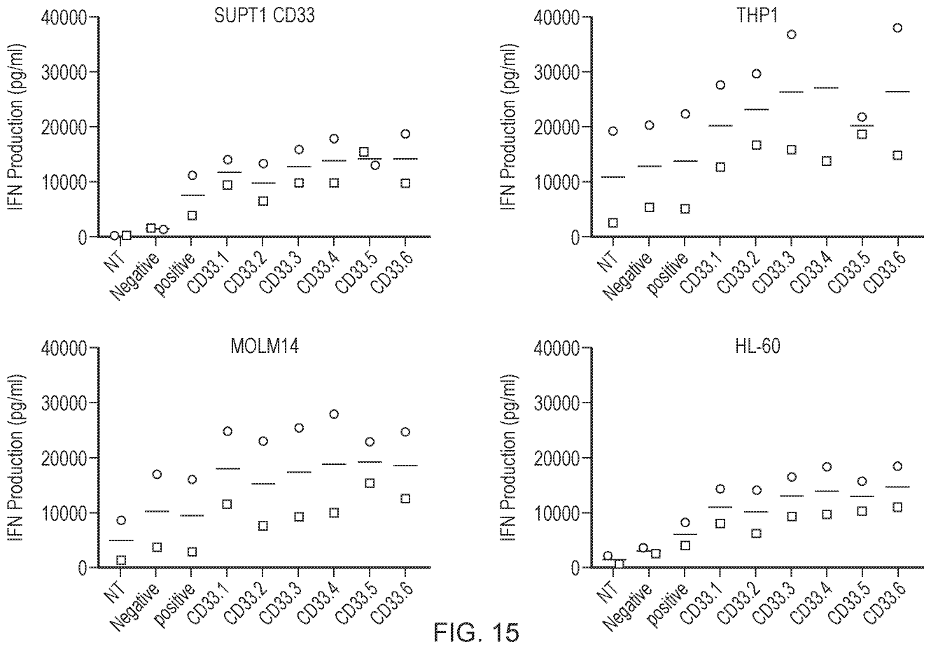

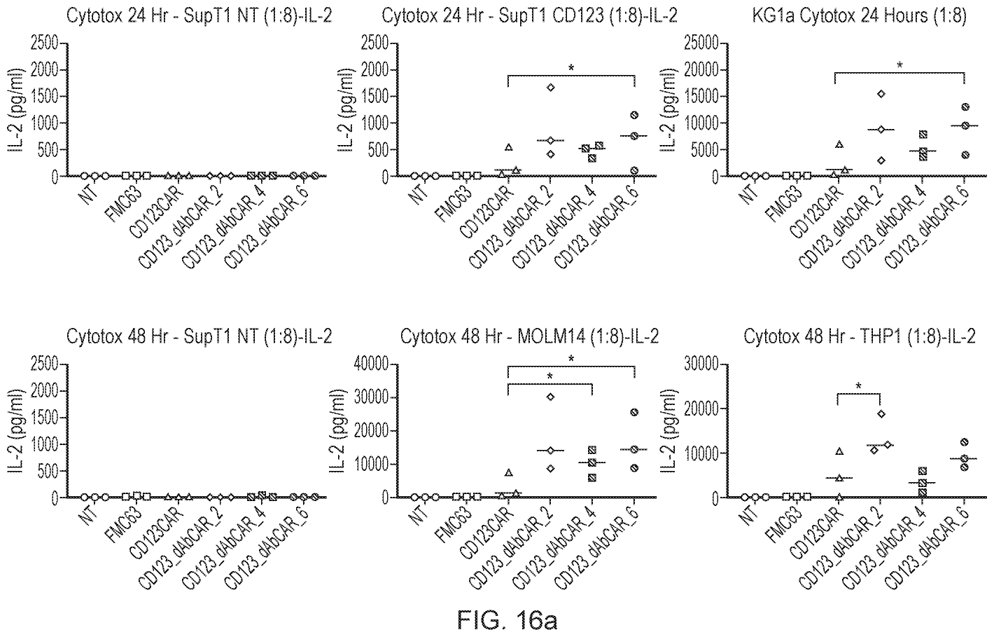

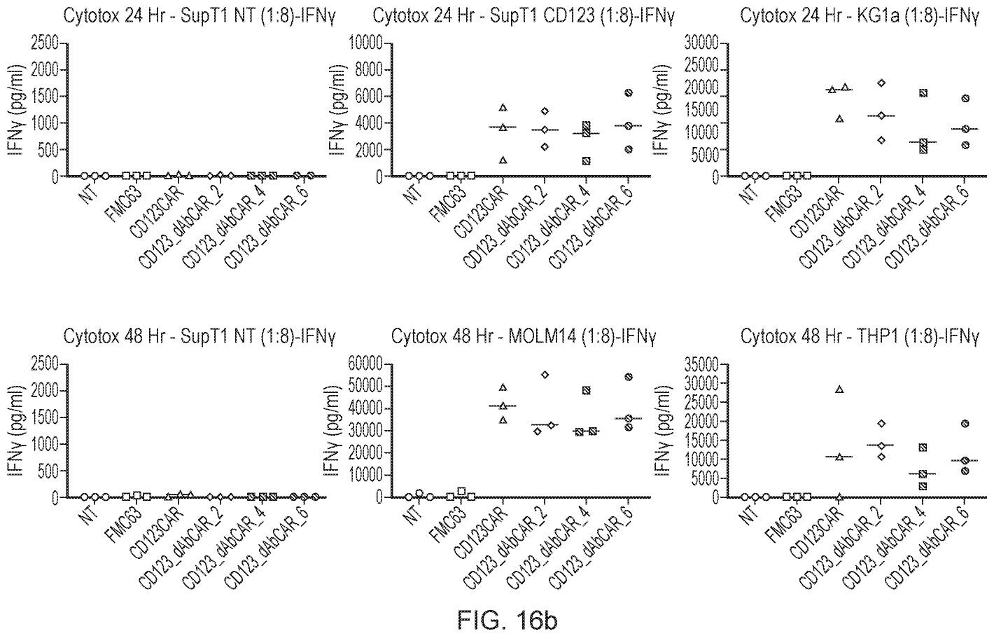

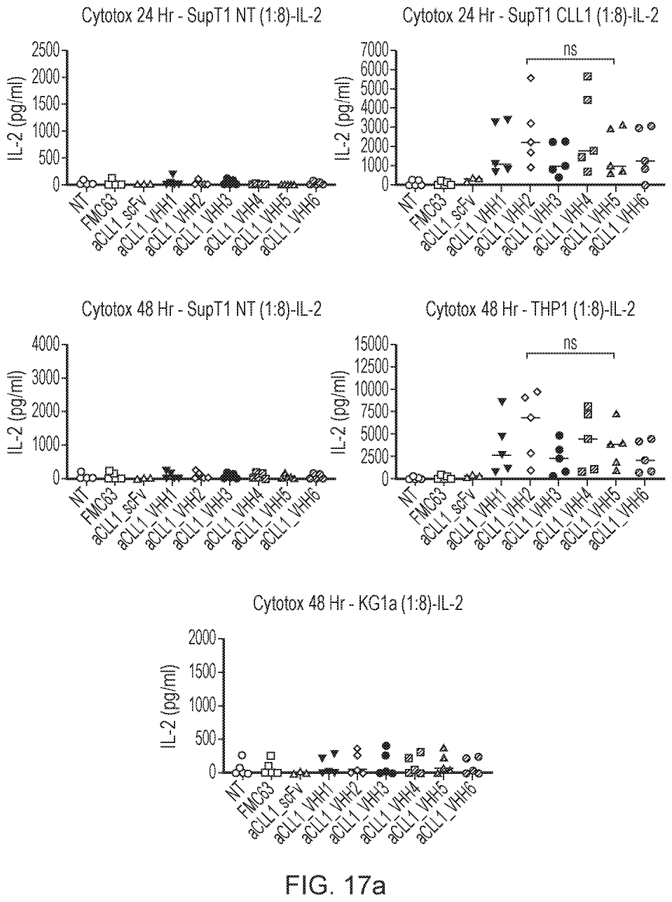

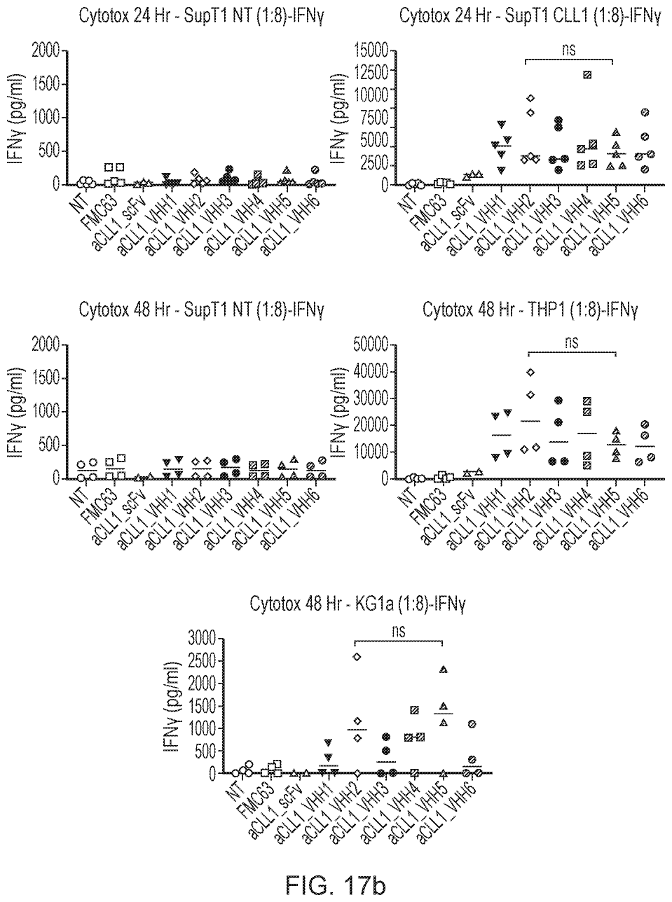

The results of the cytokine release assay are shown in FIGS. 14 and 15 for aCD33, FIG. 16 aCD123, and FIG. 17 for aCLL1.

A higher secretion of IL-2 was observed when the aCD33 CAR T cells were exposed to the CD33 positive cells. All aCD33 sdAb CAR T cells produced relatively more IL-2 than non-transduced and negative control. The IL-2 secretion level was comparatively lower with aCD33 scFv positive control. Similar level of IL-2 production was observed with FMC63 negative control compared with the aCD33 scFv positive control. IFNy production was significantly higher for a aCD33 CAR T cells than negative control. All aCD33 sdAb CARs produced similar level of IFN-γ in both engineered SupT1 cells and AML derived cells (FIG. 15). sdAb CD33.6 maintained the IFN-γ production in greater level in all cells. Whereas sdAb CD33.2IFN-γ production was lower compared with all other sdAb CD33 CARs.

IFNγ levels produced by aCD33 scFv were lower when challenged with THP1 and MOLM14 cells. Not much of a difference in IFN-γ production was observed between the engineered SupT1 and AML derived cells. FMC63 scFv negative control also exhibited to produce the same level of IFN-γ (^{˜}10000 pg/ml) compared with the aCD33 scFv positive control on MOLM14 cells.

All aCD123 CAR T cells produced IL-2 when exposed to CD123+ cells but there was no significant difference for IL-2 production between VHH-CAR-T cell constructs. VHH-CAR-2 and 6 produced the highest levels of IL-2 production with Molm14 (mean ˜1.77×104 and 1.63×104 pg/ml), and VHH-CAR-4 produced the lowest IL-2 production with THP1 (mean -3.5x103 pg/ml). The VHH-CAR-constructs displayed improved IL-2 production when compared to the scFv CAR positive control, both for the 24 and 48hr time course even though no significant difference was seen for the cytotoxicity assay.

All aCD123 CARs produce similar levels of IFNγ against SupT1 CD123 and Molm14 (mean ˜3.4×103, and ˜4.0×104 pg/ml, respectively). There was no significant difference between CD123 CAR constructs for IFNγ production across all constructs. As with IL-2 production, VHH-CAR-6 consistently produced greater levels of cytokines across all conditions, although there was no significant difference. The scFv control CAR produced similar levels of IFNγ as the VHH CARs agreeing with the trend seen for target lysis in the cytotoxicity assay. The highest IFNγ production was for VHH-CAR-6 for Molm14 (mean ≠4.0×104 pg/ml) whilst the lowest was for VHH-CAR-4 with THP1 (mean ≠1.45×103 pg/ml.

Antigen-specific IL-2 production was observed for all CLL1-VHH-CAR constructs with SupT1 CLL1 and THP1, but no significant difference was observed between constructs (FIG. 17,a). However, there was no antigen-specific IL-2 production for CARs co-cultured with KG1a even though killing data does display antigen-specific cytotoxicity. This observation could be linked to the very low expression level of CLL1 on the cell surface of KG1a cells (638/cell, FIG. 7). A similar trend was seen for IFNγ production, where all CAR constructs displayed antigen-specific IFNγ production for SupT1 CLL1 and THP1, without significant difference between constructs (FIG. 17, b). Again, relative low levels of IFNγ were produced for KG1a, with CARs that performed well for the cytotoxicity assay producing greater levels of cytokines (VHH-CAR 2, 4, and 5).

### Example 4—Proliferation Assay (PA)

In order to measure proliferation, the same panel of CAR-expressing T cells described in Example 1 were labelled with the dye Cell Trace Violet (CTV), a fluorescent dye which is hydrolysed and retained within the cell. It is excited by the 405 nm (violet) laser and fluorescence can be detected in the pacific blue channel.

The CTV dye was reconstituted to 5 mM in DMSO. The T-cells were resuspended at 2×106 cells per ml in PBS, and 1 ul/ml of CTV was added. The T-cells were incubated the CTV for 20 minutes at 37° C. Subsequently, the cells were quenched by adding 5V of complete media. After a 5 minutes incubation, the T-cells were washed and resuspended in 2 ml of complete media. An additional 10 minute incubation at room temperature allows the occurrence of acetate hydrolysis and retention of the dye.

Labelled T-cells were co-cultured with target cells for four days. The assay was carried out in a 96-well plate in 0.2 ml total volume using 5×10⁴ transduced T-cells per well and an equal number of target cells (ratio 1:1). At the day four time point, the T-cells were analysed by flow cytometry to measure the dilution of the CTV which occurs as the T-cells divide. The number of T-cells present at the end of the co-culture was calculated, and expressed as a fold of proliferation compared to the input number of T cells.

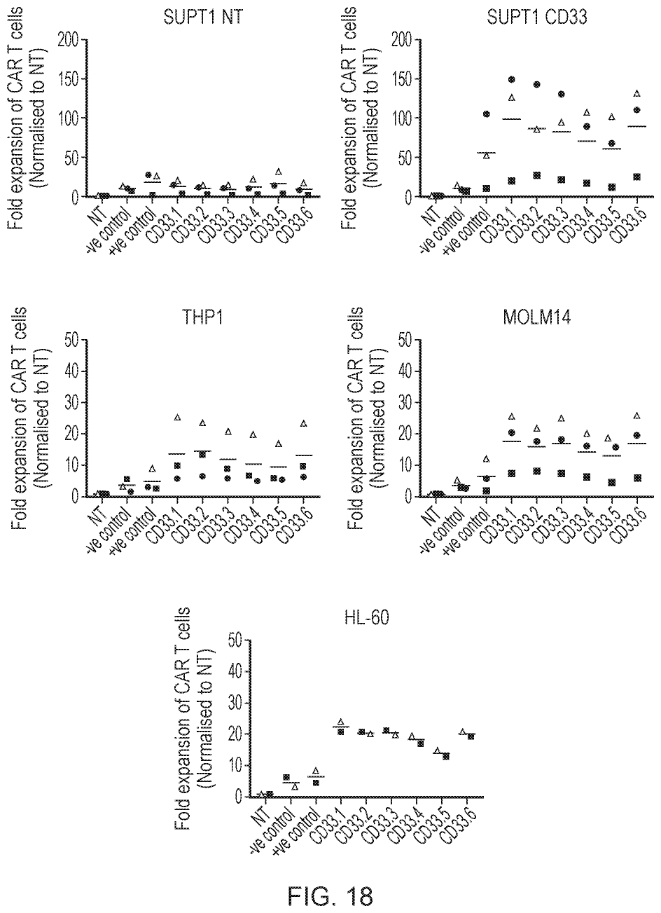

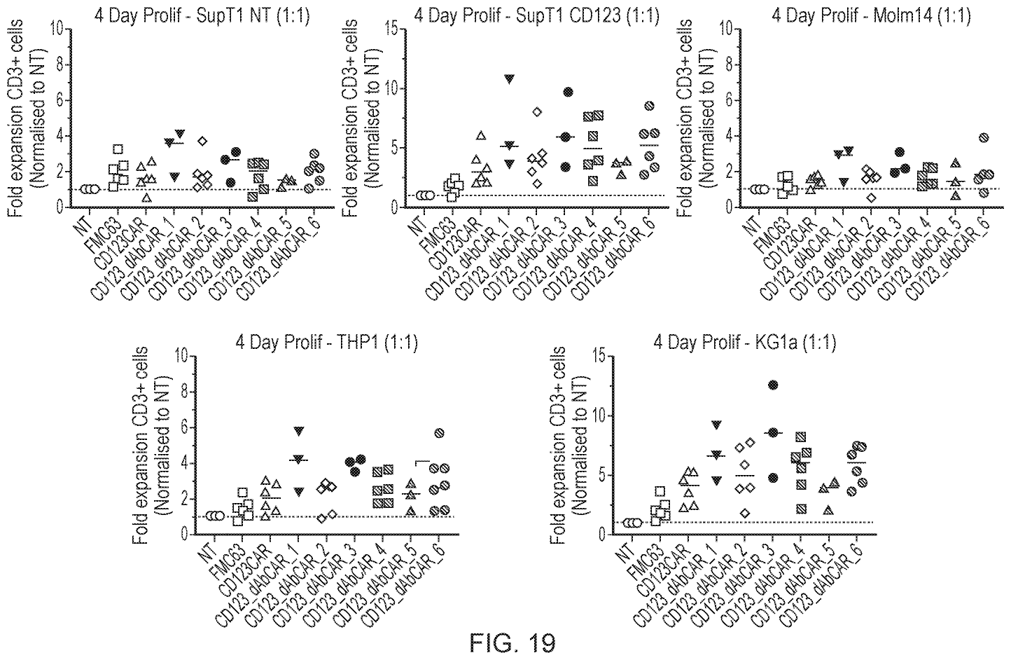

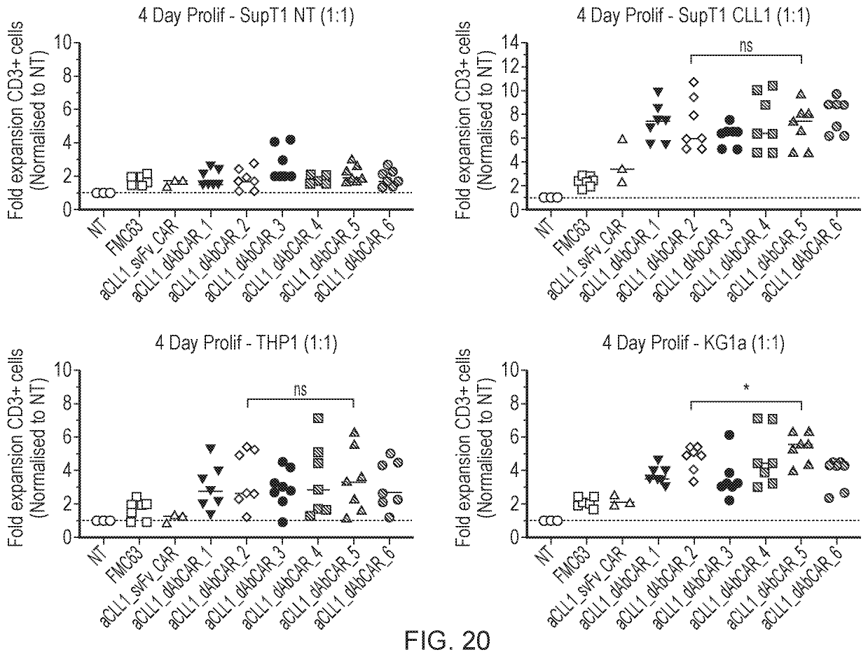

The results of the proliferation assay are shown in FIG. 18 for aCD33, FIG. 19 aCD123, and FIG. 20 for aCLL1.

There was a significant difference in the fold expansion of aCD33 CAR T cells observed after 6 days. Higher fold expansion was observed with aCD33 sdAb CAR T cells compared with the non—transduced control and negative control (FMC63 scFv). It should be noted that the proliferation level of the positive control (aCD33 scFv) was lower compared with the aCD33 sdAb CAR T cells in all AML cells. The fold expansion of CAR T cells was significantly higher in SupT1 artificially transduced with CD33 comparing with other cells.

No significant difference was observed between all CD123-VHH-CAR constructs across all conditions.

Antigen-specific proliferation was observed for all CLL1-VHH-CAR constructs with CLL1+ target cell lines, however, there was no significant difference observed for proliferative capacity with SupT1 CLL1 and THP1. For KG1a co-cultures, there was significant proliferative advantage for CLL1-VHH-CARs 2 and 5 over CLL1-VHH-CARs 1, 2, and 3 across all ratios (p values range from <0.05 to <0.01, n=7). With CLL1-VHH-CAR-5 displaying significant proliferative advantage over CLL1-VHH-CAR-2 with KG1a (p=0.0445, n=7).

All six aCD33 single dab CAR exhibited a similar levels of response with target cells in all proliferation, cytotoxicity, and cytokine production assays compared with the negative CAR control (FMC63 scFv). sdAb CD123 CARs showed efficacy against engineered SupT1 CD123 cells and other AML derived cells (MOLM14, THP1, KG1a) in a dose-response manner. sdAb CLL1 CAR was also effective against CLL1+cells. The results obtained from the first phase of the study demonstrated each single domain binder (CD123, CD33, and CLL1) worked efficiently as a second-generation CARs against AML.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A cell comprising: an anti-CD33 chimeric antigen receptor antibody antigen-binding domain (dAbCAR); an anti-CLL1 dAbCAR; and an anti-CD123 CAR.

2-7. (canceled)

8. A cell according to claim 1 wherein the anti-CD33 dAbCAR has an antigen binding domain comprising the following complementarity determining regions:
(i)
CDR1
(SEQ ID No. 1)
GRTFSMHS;
CDR2
(SEQ ID No. 2)
VTWSGDTF;
CDR3
(SEQ ID No. 3)
KDDPYRPAYDY;
(ii)
CDR1
(SEQ ID No. 4)
GRTFSSYV;
CDR2
(SEQ ID No. 5)
ISWSGGST;
CDR3
(SEQ ID No. 6)
AAMELRGGSYNYASSRQYDY;
(iii)
CDR1
(SEQ ID No. 7)
EIAFSNFN;
CDR2
(SEQ ID No. 8)
ISSHGDTNY;
CDR3
(SEQ ID No. 9)
NANDPFLSVSDF;
(iv)
CDR1
(SEQ ID No. 10)
GSIFSINA;
CDR2
(SEQ ID No. 5)
ISWSGGST;
CDR3
(SEQ ID No. 11)
AAISGWGRSIRVGERYEYDY;
(v)
CDR1
(SEQ ID No. 12)
GRTSSSST;
CDR2
(SEQ ID No. 13)
ITLSGGST;
CDR3
(SEQ ID No. 14)
AARRWSNNRGGYDRAGYDY;
or
(vi)
CDR1
(SEQ ID No. 15)
GRTFSSYA;
CDR2
(SEQ ID No. 16)
ITWSGGST;
CDR3
(SEQ ID No. 17)
AMLLRGGLYDYTDYILYNY.

9. A cell according to claim 8, wherein the antigen binding domain comprises one of the sequences shown as SEQ ID No. 18, 19, 20, 21, 22 or 23.

10. A cell according to claim 1 wherein the anti-CLL1 dAbCAR has a an antigen binding domain comprising the following complementarity determining regions:
(i)
CDR1
(SEQ ID No. 48)
GFTFGNHD;
CDR2
(SEQ ID No. 49)
IDSGGNVI;
CDR3
(SEQ ID No. 50)
ATDLDSGAESLESVY;
(ii)
CDR1
(SEQ ID No. 51)
GFAFGSAD;
CDR2
(SEQ ID No. 52)
IDSGGNTQ;
CDR3
(SEQ ID No. 53)
TDLDPTTDSLENVY;
(iii)
CDR1
(SEQ ID No. 54)
GRTFSAYF;
CDR2
(SEQ ID No. 55)
INWNGDSS;
CDR3
(SEQ ID No. 56)
AADTHGAVGLGSERLYDY;
(iv)
CDR1
(SEQ ID No. 57)
GIGVSSTG;
CDR2
(SEQ ID No. 58)
IDRDGTT;
CDR3
(SEQ ID No. 59)
TVVGDYY;
(v)
CDR1
(SEQ ID No. 60)
GFIFGNYD;
CDR2
(SEQ ID No. 61)
ISSGGNDI;
CDR3
(SEQ ID No. 62)
AADLDPGTDSLDNIH;
or
(vi)
CDR1
(SEQ ID No. 63)
GFTLDYYA;
CDR2
(SEQ ID No. 64)
ISSSDGST;
CDR3
(SEQ ID No. 65)
AEAVYYAGVCVAMYDS.

11. A cell according to claim 10, wherein the antigen binding domain comprises one of the sequences shown as SEQ ID No. 66, 67, 68, 69, 70 or 71.

12. A cell according to claim 1 wherein the anti-CD123 dAbCAR has an antigen binding domain comprising the following complementarity determining regions:
(i)
CDR1
(SEQ ID No. 24)
GRSINTYA;
CDR2
(SEQ ID No. 25)
INYNSRYT;
CDR3
(SEQ ID No. 26)
AATSYYPTDYDVASRVATWPS;
(ii)
CDR1
(SEQ ID No. 27)
GISLNA;
CDR2
(SEQ ID No. 28)
IKIGGVS;
CDR3
(SEQ ID No. 29)
NTYPPYLNGMDY;
(iii)
CDR1
(SEQ ID No. 30)
GRSFNTDA;
CDR2
(SEQ ID No. 31)
ISWDGTRT;
CDR3
(SEQ ID No. 32)
AAEPQKAWPIGTSAAGFRS;
(iv)
CDR1
(SEQ ID No. 33)
GSSISV;
CDR2
(SEQ ID No. 34)
ISWSDGNT;
CDR3
(SEQ ID No. 35)
AVEPRGWPKGHRY;
(v)
CDR1
(SEQ ID No. 36)
GSSFSINV;
CDR2
(SEQ ID No. 37)
ISWSDGST;
CDR3
(SEQ ID No. 38)
AVEPRGWPKGHRY;
or
(vi)
CDR1
(SEQ ID No. 39)
GSIFRINA;
CDR2
(SEQ ID No. 40)
VNWIGGTT;
CDR3
(SEQ ID No. 41)
SATDKGGSSRY.

13. A cell according to claim 12, wherein the antigen binding domain comprises one of the sequences shown as SEQ ID No. 42, 43, 44, 45, 46 or 47.

14-15. (canceled)

16. A nucleic acid construct encoding: an anti-CD33 chimeric antigen receptor comprising a domain antibody antigen-binding domain (dAbCAR); an anti-CLL1 dAbCAR; and an anti-CD123 dAbCAR.

17-18. (canceled)

19. A method for making a cell according to claim 1 which comprises the step of transducing or transfecting a cell with a nucleic acid construct encoding: an anti-CD33 dAbCAR; an anti-CLL1 dAbCAR; and an anti-CD123 dAbCAR.

20. (canceled)

21. A vector comprising a nucleic acid construct according to claim 16.

22. A kit of vectors, which comprises:
(i) a first vector which comprises a nucleic acid sequence encoding a chimeric antigen receptor comprising a domain antibody antigen-binding domain (dAbCAR) which binds CD33;
(ii) a second vector which comprises a nucleic acid sequence encoding a dAbCAR which binds CLL1; and
(iii) a third vector which comprises a nucleic acid sequence encoding a dAbCAR which binds CD123.

23. (canceled)

24. A pharmaceutical composition which comprises a plurality of cells according claim 1, together with a pharmaceutically acceptable carrier, diluent or excipient.

25. A method for treating cancer which comprises the step of administering a pharmaceutical composition according to claim 24 to a subject.

26. A method according to claim 25, wherein the cancer is acute myeloid leukemia (AML).

27. A method according to claim 26, which also involves the step of subsequently administering an allogeneic transplant to the subject.

28-29. (canceled)


SEQUENCE LISTING (95 sequences)

SEQ ID NO: 1 - protein, 8 aa, Artificial Sequence
  anti-CD33 chimeric antigen receptor (CAR) complementarity determining region (CDR) CDR1
GRTFSMHS

SEQ ID NO: 2 - protein, 8 aa, Artificial Sequence
  anti-CD33 CAR, CDR2
VTWSGDTF

SEQ ID NO: 3 - protein, 11 aa, Artificial Sequence
  anti-CD33 CAR, CDR3
KDDPYRPAYDY

SEQ ID NO: 4 - protein, 8 aa, Artificial Sequence
  anti-CD33 CAR, CDR1
GRTFSSYV

SEQ ID NO: 5 - protein, 8 aa, Artificial Sequence
  anti-CD33 CAR, CDR2
ISWSGGST

SEQ ID NO: 6 - protein, 20 aa, Artificial Sequence
  anti-CD33 CAR, CDR3
AAMELRGGSYNYASSRQYDY

SEQ ID NO: 7 - protein, 8 aa, Artificial Sequence
  anti-CD33 CAR, CDR1
EIAFSNFN

SEQ ID NO: 8 - protein, 9 aa, Artificial Sequence
  anti-CD33 CAR, CDR2
ISSHGDTNY

SEQ ID NO: 9 - protein, 12 aa, Artificial Sequence
  anti-CD33 CAR, CDR3
NANDPFLSVSDF

SEQ ID NO: 10 - protein, 8 aa, Artificial Sequence
  anti-CD33 CAR, CDR1
GSIFSINA

SEQ ID NO: 11 - protein, 20 aa, Artificial Sequence
  anti-CD33 CAR, CDR3
AAISGWGRSIRVGERYEYDY

SEQ ID NO: 12 - protein, 8 aa, Artificial Sequence
  anti-CD33 CAR, CDR1
GRTSSSST

SEQ ID NO: 13 - protein, 8 aa, Artificial Sequence
  anti-CD33 CAR, CDR2
ITLSGGST

SEQ ID NO: 14 - protein, 19 aa, Artificial Sequence
  anti-CD33 CAR, CDR3
AARRWSNNRGGYDRAGYDY

SEQ ID NO: 15 - protein, 8 aa, Artificial Sequence
  anti-CD33 CAR, CDR1
GRTFSSYA

SEQ ID NO: 16 - protein, 8 aa, Artificial Sequence
  anti-CD33 CAR, CDR2
ITWSGGST

SEQ ID NO: 17 - protein, 19 aa, Artificial Sequence
  anti-CD33 CAR, CDR3
AMLLRGGLYDYTDYILYNY

SEQ ID NO: 18 - protein, 119 aa, Artificial Sequence
  CD33 domain antibody (dAb) sequence, CD33 dAb P1.E4 - 44738
QVQLESGGGLVQAGGSLRLSCAASGRTFSMHSMGWFRQAPGKEREFVAAVTWSGDTFAYADFVKGRFTISRGIAKNTLYLQMNSLKPEDTAVYYCAAKDDPYRPAYDYWGQGTQVTVSS

SEQ ID NO: 19 - protein, 127 aa, Artificial Sequence
  CD33 dAb sequence, CD33 dAb P1.H3- 44739
QVQLQESGGGLVQAGGSLRLSCAASGRTFSSYVMGWFRQAPGKEREFVAAISWSGGSTYYADSVKGRFTISRDNAKNTLYLQMNSLKPEDTAVYYCAAMELRGGSYNYASSRQYDYWGQGTQVTVSS

SEQ ID NO: 20 - protein, 118 aa, Artificial Sequence
  CD33 dAb sequence, CD33 dAb P1.G8 - 44742
QVQLQESGGGLVQTGGSLTLSCAASEIAFSNFNMGWYRQGSGKQRTLVAQISSHGDTNYLDSMKGRFTISRDNNKKTVYLQMNALKPEDTAVYYCNANDPFLSVSDFWGQGTQVTVSS

SEQ ID NO: 21 - protein, 127 aa, Artificial Sequence
  CD33 dAb sequence, CD33 dAb P2.A7 - 46173
QVQLQQSGGGLVQAGGSLRLSCAASGSIFSINAMGWFRQAPGKEREFVAAISWSGGSTYYADFVKGRFTISRDNAKNTVYLQMNSLKPEDTAIYYCAAISGWGRSIRVGERYEYDYWGQGTQVTVSS

SEQ ID NO: 22 - protein, 126 aa, Artificial Sequence
  CD33 dAb sequence, CD33 dAb P2.B12 - 46174
QVQLQESGGGLVQAGGSLRLSCAASGRTSSSSTMAWFRQAPGKEREFVAAITLSGGSTHYADSAKGRFTISRESAKNTVYLQMNSLKPEDTADYYCAARRWSNNRGGYDRAGYDYWGQGTQVTVSS

SEQ ID NO: 23 - protein, 127 aa, Artificial Sequence
  CD33 dAb sequence, CD33 dAb P2.F2 - 46176
QVQLQESGGGLVQAGGSLRLSCAASGRTFSSYAMGWFRQAPGKEREFVAAITWSGGSTYYADSVKGRFTISRDNAKNTLYLQMNSLKPEDTAVYYCAAMLLRGGLYDYTDYILYNYWGQGTQVTVSS

SEQ ID NO: 24 - protein, 8 aa, Artificial Sequence
  anti-CD123 CAR, CDR1
GRSINTYA

SEQ ID NO: 25 - protein, 8 aa, Artificial Sequence
  anti-CD123 CAR, CDR2
INYNSRYT

SEQ ID NO: 26 - protein, 21 aa, Artificial Sequence
  anti-CD123 CAR, CDR3
AATSYYPTDYDVASRVATWPS

SEQ ID NO: 27 - protein, 6 aa, Artificial Sequence
  anti-CD123 CAR, CDR1
GISLNA

SEQ ID NO: 28 - protein, 7 aa, Artificial Sequence
  anti-CD123 CAR, CDR2
IKIGGVS

SEQ ID NO: 29 - protein, 12 aa, Artificial Sequence
  anti-CD123 CAR, CDR3
NTYPPYLNGMDY

SEQ ID NO: 30 - protein, 8 aa, Artificial Sequence
  anti-CD123 CAR, CDR1
GRSFNTDA

SEQ ID NO: 31 - protein, 8 aa, Artificial Sequence
  anti-CD123 CAR, CDR2
ISWDGTRT

SEQ ID NO: 32 - protein, 19 aa, Artificial Sequence
  anti-CD123 CAR, CDR3
AAEPQKAWPIGTSAAGFRS

SEQ ID NO: 33 - protein, 6 aa, Artificial Sequence
  anti-CD123 CAR, CDR1
GSSISV

SEQ ID NO: 34 - protein, 8 aa, Artificial Sequence
  anti-CD123 CAR, CDR2
ISWSDGNT

SEQ ID NO: 35 - protein, 13 aa, Artificial Sequence
  anti-CD123 CAR, CDR3
AVEPRGWPKGHRY

SEQ ID NO: 36 - protein, 8 aa, Artificial Sequence
  anti-CD123 CAR, CDR1
GSSFSINV

SEQ ID NO: 37 - protein, 8 aa, Artificial Sequence
  anti-CD123 CAR, CDR2
ISWSDGST

SEQ ID NO: 38 - protein, 13 aa, Artificial Sequence
  anti-CD123 CAR, CDR3
AVEPRGWPKGHRY

SEQ ID NO: 39 - protein, 8 aa, Artificial Sequence
  anti-CD123 CAR, CDR1
GSIFRINA

SEQ ID NO: 40 - protein, 8 aa, Artificial Sequence
  anti-CD123 CAR, CDR2
VNWIGGTT

SEQ ID NO: 41 - protein, 11 aa, Artificial Sequence
  anti-CD123 CAR, CDR3
SATDKGGSSRY

SEQ ID NO: 42 - protein, 128 aa, Artificial Sequence
  CD123 dAb sequence, CD123 dAb H11 45897
QVQLQESGGGLVQAGGSLRLSCTASGRSINTYAMAWFRQAPGKEREFVASINYNSRYTHYVDSVKGRFTISRDNTKNTLFLQMDSLNREDTAVYYCAATSYYPTDYDVASRVATWPSWGQGTQVTVSS

SEQ ID NO: 43 - protein, 117 aa, Artificial Sequence
  CD123 dAb sequence, CD123 dAb F8 45888
QVQLQESGGGLVQAGESLRLTCAVSGISLNAMGWYRQAPGKQLREWVAVIKIGGVSNYAVSVKGRFTISRDNAKNTIYLQMNSLKPEDTGVYYCNTYPPYLNGMDYWGKGTLVTVSS

SEQ ID NO: 44 - protein, 126 aa, Artificial Sequence
  CD123 dAb sequence, CD123 dAb A7 45865
QVQLQQSGGGLVQAGGSLRLSCAFSGRSFNTDAVAWFRQAPGKEREFVAAISWDGTRTYYADSAKGRFTISRDNAKNTVYLQMNSLNSEDTAVYYCAAEPQKAWPIGTSAAGFRSWGQGTQVTVSS

SEQ ID NO: 45 - protein, 118 aa, Artificial Sequence
  CD123 dAb sequence, CD123 dAb B4 45868
QVQLQESGGGSVQSGGSLRLSCAASGSSISVMGWFRQAPGKEREFVAAISWSDGNTNYADSVNGRFSVSRDNTKNTVYLQMNSLKPEDTAIYYCAVEPRGWPKGHRYWGQGTQVTVSS

SEQ ID NO: 46 - protein, 120 aa, Artificial Sequence
  CD123 dAb sequence, CD123 dAb A10 45866
QVQLQESGGSSVQAGGSLRLSCAASGSSFSINVMGWFRQAPGKEREFVAAISWSDGSTNYADSVKGRFTISRDNTKNTVYLQMNSLKPEDTAIYYCAVEPRGWPKGHRYWGQGTQVTVSS

SEQ ID NO: 47 - protein, 118 aa, Artificial Sequence
  CD123 dAb sequence, CD123 dAb C11 45874
QVQLQESGGGLVQAGGSLRLSCAASGSIFRINAMGWFRQAPGKEREFVTAVNWIGGTTNYADSVKGRFTISRDNAKNTVYLQMNSLKPEDTAIYFCSATDKGGSSRYWGQGTQVTVSS

SEQ ID NO: 48 - protein, 8 aa, Artificial Sequence
  anti-CLL-1 CAR, CDR1
GFTFGNHD

SEQ ID NO: 49 - protein, 8 aa, Artificial Sequence
  anti-CLL-1 CAR, CDR2
IDSGGNVI

SEQ ID NO: 50 - protein, 15 aa, Artificial Sequence
  anti-CLL-1 CAR, CDR3
ATDLDSGAESLESVY

SEQ ID NO: 51 - protein, 8 aa, Artificial Sequence
  anti-CLL-1 CAR, CDR1
GFAFGSAD

SEQ ID NO: 52 - protein, 8 aa, Artificial Sequence
  anti-CLL-1 CAR, CDR2
IDSGGNTQ

SEQ ID NO: 53 - protein, 14 aa, Artificial Sequence
  anti-CLL-1 CAR, CDR3
TDLDPTTDSLENVY

SEQ ID NO: 54 - protein, 8 aa, Artificial Sequence
  anti-CLL-1 CAR, CDR1
GRTFSAYF

SEQ ID NO: 55 - protein, 8 aa, Artificial Sequence
  anti-CLL-1 CAR, CDR2
INWNGDSS

SEQ ID NO: 56 - protein, 18 aa, Artificial Sequence
  anti-CLL-1 CAR, CDR3
AADTHGAVGLGSERLYDY

SEQ ID NO: 57 - protein, 8 aa, Artificial Sequence
  anti-CLL-1 CAR, CDR1
GIGVSSTG

SEQ ID NO: 58 - protein, 7 aa, Artificial Sequence
  anti-CLL-1 CAR, CDR2
IDRDGTT

SEQ ID NO: 59 - protein, 7 aa, Artificial Sequence
  anti-CLL-1 CAR, CDR3
TVVGDYY

SEQ ID NO: 60 - protein, 8 aa, Artificial Sequence
  anti-CLL-1 CAR, CDR1
GFIFGNYD

SEQ ID NO: 61 - protein, 8 aa, Artificial Sequence
  anti-CLL-1 CAR, CDR2
ISSGGNDI

SEQ ID NO: 62 - protein, 15 aa, Artificial Sequence
  anti-CLL-1 CAR, CDR3
AADLDPGTDSLDNIH

SEQ ID NO: 63 - protein, 8 aa, Artificial Sequence
  anti-CLL-1 CAR, CDR1
GFTLDYYA

SEQ ID NO: 64 - protein, 8 aa, Artificial Sequence
  anti-CLL-1 CAR, CDR2
ISSSDGST

SEQ ID NO: 65 - protein, 16 aa, Artificial Sequence
  anti-CLL-1 CAR, CDR3
AEAVYYAGVCVAMYDS

SEQ ID NO: 66 - protein, 122 aa, Artificial Sequence
  CLL-1 dAb sequence, CLL-1 dAb 44548
QVQLQQSGGGLVQPGGSLRLSCVGSGFTFGNHDMSWVRQAPGKEVEFVAGIDSGGNVIVYEEVVKGRFTISRDNAKNTLYLQMDGLKPEDAGMYFCATDLDSGAESLESVYHGQGTQVTVSS

SEQ ID NO: 67 - protein, 122 aa, Artificial Sequence
  CLL-1 dAb sequence, CLL-1 dAb 44544
QVQLQESGGGLVESGGSLRISCTGFGFAFGSADMSWVRQAPGKEVEFVAGIDSGGNTQTYEDTVKGRFTISRDNAKNTLYLQMNSLQSEDAGVYFCATDLDPTTDSLENVYHGQGTQVIVSS

SEQ ID NO: 68 - protein, 125 aa, Artificial Sequence
  CLL-1 dAb sequence, CLL-1 dAb 44538
QVQLQESGGGLVQTGDSLRLSCVASGRTFSAYFMGWFRQAPGKEREFVSAINWNGDSSWYRDSVKGRFTVSRDNAKNTVYLQMNSLEPEDTAVYYCAADTHGAVGLGSERLYDYWGQGTQVTVSS

SEQ ID NO: 69 - protein, 113 aa, Artificial Sequence
  CLL-1 dAb sequence, CLL-1 dAb 44546
QVQLQESGGGVVQAGGSLRLSCAVSGIGVSSTGMGWSRQTPGKQVELVALIDRDGTTNYADTVKGRFTISKDNSKNMVYLQMNSLKPEDTALYHCTVVGDYYWGQGTQVTVSS

SEQ ID NO: 70 - protein, 122 aa, Artificial Sequence
  CLL-1 dAb sequence, CLL-1 dAb 44545
QVQLQQSGGGLVQPGGSLRLSCVGSGFIFGNYDMSWVRQAPGKEVEFVAGISSGGNDIVYEDAVKGRFSISRDNARNTVYLDMASVKPEDAGVYYCAADLDPGTDSLDNIHHGQGTQVFVSS

SEQ ID NO: 71 - protein, 123 aa, Artificial Sequence
  CLL-1 dAb sequence, CLL-1 dAb 44536
QVQLQESGGGLVQPGGSLRLSCAASGFTLDYYAIGWFRQAPGKEREGVSCISSSDGSTAYADSVKGRFTISRDNAKNSVYLQMNSLKPEDTAVYYCAEAVYYAGVCVAMYDSWGQGTQVTVSS

SEQ ID NO: 72 - protein, 7 aa, Artificial Sequence
  anti-FLT3 CAR, CDR1
GIFKTNY

SEQ ID NO: 73 - protein, 7 aa, Artificial Sequence
  anti-FLT3 CAR, CDR2
FTNDGST

SEQ ID NO: 74 - protein, 5 aa, Artificial Sequence
  anti-FLT3 CAR, CDR3
YGLGH

SEQ ID NO: 75 - protein, 8 aa, Artificial Sequence
  anti-FLT3 CAR, CDR1
GTISSIRY

SEQ ID NO: 76 - protein, 7 aa, Artificial Sequence
  anti-FLT3 CAR, CDR2
ITSSGNT

SEQ ID NO: 77 - protein, 5 aa, Artificial Sequence
  anti-FLT3 CAR, CDR3
YTMGY

SEQ ID NO: 78 - protein, 7 aa, Artificial Sequence
  anti-FLT3 CAR, CDR1
GIFSTNY

SEQ ID NO: 79 - protein, 7 aa, Artificial Sequence
  anti-FLT3 CAR, CDR2
FTNDGGT

SEQ ID NO: 80 - protein, 5 aa, Artificial Sequence
  anti-FLT3 CAR, CDR3
CGLGH

SEQ ID NO: 81 - protein, 8 aa, Artificial Sequence
  anti-FLT3 CAR, CDR1
GSISSIRY

SEQ ID NO: 82 - protein, 7 aa, Artificial Sequence
  anti-FLT3 CAR, CDR2
ITSSGST

SEQ ID NO: 83 - protein, 5 aa, Artificial Sequence
  anti-FLT3 CAR, CDR3
YTMGY

SEQ ID NO: 84 - protein, 7 aa, Artificial Sequence
  anti-FLT3 CAR, CDR1
GIFSTNH

SEQ ID NO: 85 - protein, 7 aa, Artificial Sequence
  anti-FLT3 CAR, CDR2
FTNDGST

SEQ ID NO: 86 - protein, 5 aa, Artificial Sequence
  anti-FLT3 CAR, CDR3
YGLGH

SEQ ID NO: 87 - protein, 150 aa, Artificial Sequence
  FLT3 dAb sequence, FLT3 dAb B5
QVQLQQSGGGLVQAGGSLRLSCAASGIFKTNYMAWYRQAPGKQRELVAAFTNDGSTLYGDSVKGRFTISRDDAKYTVSLQMNSLKPEDTAVYYCYGLGHWGQGTQVIVSSEPKTPKPQPAAADDDDKEQKLISEEDLNGAAHHHHHHGAA

SEQ ID NO: 88 - protein, 151 aa, Artificial Sequence
  FLT3 dAb sequence, FLT3 dAb G3
QVQLQESGGGLVQAGGSLRLSCAASGTISSIRYMNWYRQAPGKQREVVAYITSSGNTNYADSVKGRFTISRDNAKNTVYLQMDNLKPEDTAAYYCYTMGYWGQGTQVTVSSEPKIPQPQPAAADDDDKEQKLISEEDLNGAAHHHHHHGAA

SEQ ID NO: 89 - protein, 150 aa, Artificial Sequence
  FLT3 dAb sequence, FLT3 dAb H5
QVQLQESGGGLVQAGGSLRLSCAASGIFSTNYMVWCRQAPGKQRELVAAFTNDGGTLYADSLKGRFSISQDNAKNTVLLLMNSLKPEDTAVYYCCGLGHWGRGTKVTVSSEPKIPQPQPAAADDDDKEQKLISEEDLNGAAHHHHHHGAA

SEQ ID NO: 90 - protein, 151 aa, Artificial Sequence
  FLT3 dAb sequence, FLT3 dAb D12
QVQLQESGGGLVQAGGSLRLSCAASGSISSIRYMNWYRQAPGKQRESVAWITSSGSTNYADSVQGRFTISRDNAKNTVYLQMDNLKPEDTAVYYCYTMGYWGQGTQVTVSSEPKIPQPQPAAADDDDKEQKLISEEDLNGAAHHHHHHGAA

SEQ ID NO: 91 - protein, 152 aa, Artificial Sequence
  FLT3 dAb sequence, FLT3 dAb F10
QAQVQLQESGGGLVQAGGSLRLSCAASGIFSTNHMAWYRQAPGKQRELVAAFTNDGSTLYGDSVKGRFVISRDNAKYTVFLQMNSLKPEDTAVYYCYGLGHWGQGTQVTVSSEPKTPKPQPAAADDDDKEQKLISEEDLNGAAHHHHHHGAA

SEQ ID NO: 92 - protein, 15 aa, Artificial Sequence
  hinge spacer sequence
EPKSCDKTHTCPPCP

SEQ ID NO: 93 - protein, 136 aa, Artificial Sequence
  suicide gene sequence
CPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 94 - protein, 517 aa, Artificial Sequence
  suicide gene sequence, FRB-FKBP12-L3-dCasp9
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKLEYSGGGSLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGGSGGGGSGGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 95 - protein, 20 aa, Artificial Sequence
  2A-like cleavage site sequence
RAEGRGSLLTCGDVEENPGP